(19) United States
(12) Patent Application Publication
(10) Pub. No.: US 2021/0230705 A1
(43) Pub. Date: Jul. 29, 2021

(54) METHOD TO PREDICT PATHOLOGICAL GRADE AND TO IDENTIFY DRUG TARGETS AGAINST GLIOMA TUMOR

(71) Applicant: Council of Scientific & Industrial Research, New Delhi (IN)

(72) Inventors: Ram Rup Sarkar, Pune (IN); Saikat Chowdhury, Pune (IN)

(21) Appl. No.: 17/154,379

(22) Filed: Jan. 21, 2021

(30) Foreign Application Priority Data
Jan. 21, 2020 (IN) 202011002578

(51) Int. Cl. C12Q 1/6886; G16H 50/30

(52) U.S. Cl. CPC C12Q 1/6886; C12Q 2600/118; C12Q 2600/158; G16H 50/30

(57) ABSTRACT

Systems and methods for developing predictive models are provided that consider the mechanistic regulations of various bimolecular events and measure the expression of various genes, proteins, and metabolites. The predictive models are utilized in a computer-implemented method to classify the patient derived glioma tumor samples in different pathological grades and successively predict the patient specific combination of drug targets by analyzing the intra-tumor heterogeneity.

### CROSS-REFERENCES TO RELATED APPLICATIONS

This application claims the benefit of priority under 35 U.S.C. § 119 to Indian Patent Application 202011002578, filed Jan. 21, 2020, which application is hereby incorporated by reference herein in its entirety.

### TECHNICAL FIELD

The present application relates to a computer-aided systems and methods for predicting the appropriate histopathological grade (WHO recommended “low or Grade-I/II” and “high or Grade-III/IV”) of a glioma tumor by using transcriptomics data of select of genes/proteins obtained from the tumor sample.

### BACKGROUND

High grade brain tumor or Glioblastoma multiforme (GBM) is a malignant, highly lethal disease, which is commonly formed in the central nervous system (CNS) of the human brain. Glioblastoma stem cells (GSC's) are an important sub-population of tumorigenic cells found within a matured GBM tumor. These cells are mainly responsible for the development, proliferation and migration of GBM cells in human brain. GSC's which reside in the sub-ventricular zone (SVZ) of the human brain are difficult to remove by surgery and they most often escape damages caused by radiation and chemotherapeutic doses during cancer therapy.

In earlier studies it has been observed that the developmental Notch signaling pathway which is the main regulator of maintenance of adult neural stem cells (aNSCs) is also active in proliferating GSCs. In fact, researchers have found significant levels of similarities in the transcriptional states between aNSCs and GSCs. Similar to the aNSCs, GSCs reside at the top of stem cell differential hierarchy, which can undergo continuous self-renewal and differentiation processes. The existing heterogeneities of the transcriptional states between GSCs and its high proliferation rate make these cells more resistant against conventional chemo-radio therapy. It has been observed that the active Notch pathway, which confers the ability of continuous self-renewal to aNSCs, also acts in similar ways inside proliferating GSCs to retain its stemness properties for a long interval of time. In general, Notch pathway suppresses the differentiation process (neurogenesis and gliogenesis) of the stem cells by expressing bHLH (Helix-loop-helix) transcription repressor proteins HES1-7 and HEY1, 2, L. These proteins are mainly responsible for repression of genes involved in development of matured and differentiated neurons and glial (astrocytes, oligodendrocytes, etc.) cells from undifferentiated stem cells. On the contrary, constitutive activation of Notch pathway and over-expressions of its component proteins are also observed in differentiated normal astrocytes/glial cells and in differentiated GBM tumor cells. As a result, inhibition of this pathway is proven fruitful to suppress proliferations of high grade (Grade-III and IV) Glioblastoma (HGG) tumor cells.

It is clearly evident from these observations that Notch pathway plays a significant role to regulate the differential hierarchy of adult neural and glioblastoma stem cells. Therefore, understanding molecular events (physical interactions, chemical reactions, translocation, transcriptions, cellular trafficking, etc.) of this pathway in aNSCs and GSCs will be beneficial to solve various critical problems, which mostly arise during diagnosis and prognosis of brain tumor patients. Most often oncologists are required to make decisions about the future course of therapy based on the tumor grade predicted by radiologists/pathologists. Tumor grades are declared based on certain cellular and morphological features observed in radiological or histopathological (HnE or IHC) images obtained from brain tumor. The decision made about tumor grade by this process is always subjective in nature, which largely depends on the domain expertise of the respective radiologists and pathologists examining the tumor sample.

Even though various computer assisted diagnosis (CAD) systems have been widely used to reduce such subjective bias, the task of tumor grade detection has not yet been simplified. The experimental noise due to wrong calibration of instruments and staining of tumor tissue can still widely hamper diagnosis of tumor samples. Due to presence of high inter-tumor variability and intra-tumor heterogeneity in glioma, mostly in primary and secondary Grade-IV GBM tissue, oncologists face serious challenges to decide effective personalized drug-targets for individual patients. To address these current challenges, molecular marker (genetic, epigenetic) based diagnostic and prognostic approaches have drawn the attention of researchers for designing personalized therapy for individual GBM patients.

US Patent Publication No. 2019/0156159 discloses a computer implemented method for characterization of a brain tumor. The method is based on a data-driven approach to extract brain tumor information from data obtained from Whole Slide Image that is uploaded through an interface. Using the approach, information relating to glioblastoma tumor from a brain biopsy slide using neural networks: annotated areas of relevant tissues, molecular subtype is generated. It is to be noted that the features of the brain tumor are visualised based on structural analysis, however, the expression of protein markers is not ascertained therein.

High-throughput omics-based experimental assays followed by state-of-the-art bioinformatics analyses have been employed to extract important markers (genes, proteins, metabolites) and classify patients in different pathological (low and high-grade) and treatment (responsive and non-responsive) groups. However, such approaches most often detect a large number of genes/proteins, pathways, biochemical events, and cellular functions as the important factors for diagnosis and prognosis of GBM therapy. Differential expressions studies performed between two treatment groups (responsive and non-responsive) can identify a large set of genes important for treatment response, but cannot precisely pin-point the right targets appropriate for specific group of patients. It has been observed that expressions or genetic states of individual molecular markers may not always predict accurate grade and therapeutic response of all GBM tumors. The GBM tumor with amplified EGFR is not always treatable by using targeted EGFR therapy because another Receptor tyrosine kinases (RTK) protein PDGFR may be co-amplified in one of the subpopulation of tumor cells. Indeed, in such cases, dual inhibition of these RTKs with anti-EGFR and anti-PDGFR inhibitors has been found most effective therapy. Such observations have clearly proven that interpretation of appropriate pathological grade and prognostic outcomes of GBM tumor cannot be achieved by only measuring expression levels of individual biomarkers. To achieve higher accuracy, better predictive models, which can simulate and analyze cancer cell development as much as close to real biological scenarios are highly desirable for diagnosis and prognosis of GBM tumor. The predictive models which will take into account the mechanisms of various oncogenic molecular reactions or pathways as well as expressions of various genes, proteins, and metabolites of cancer cells could be the best option in this case.

There is an emergent need in the art to arrive at predictive mechanistic models, considering the dynamic activities of Notch and its cross-talks pathways for futuristic predictions of the development of neurogenesis and GBM tumorigenesis in the human brain originating from aNSCs or GSCs. Therefore, the present Applicant has provided a method for quantifying the risk of tumor development by considering genetic variances of individual patients as inputs. Moreover, the assessment of targeting multiple combinations of proteins also needs to be performed through a novel approach developed for in-silico perturbations analyses on the developed models.

Thus, an object of this application is to provide an automated system and method to detect the glioma tumor in a subject diagnosed with glioblastoma and subsequently classify the tumor sample as per the WHO recommended histopathological grade by assessing the transcriptomics or proteomics states of the select proteins associated with tumorigenesis.

Another object of this application is to provide a quantitative scoring system to compute the confidence interval and probability of the tumor sample of a human patient developing low or high-grade glioma tumor and to estimate the biological activity of different proteins highly crucial for the development of different grades of gliomas cells during tumorigenesis.

Further, an object of this application is to provide a process for the personalized screening and extracting potential drug-targets for suppressing tumor cell growth.

### SUMMARY

Accordingly, embodiments disclosed herein relate to a system and methods for developing predictive models by considering the mechanistic regulations of various biomolecular events and measuring the expressions of various genes, proteins and metabolites associated in Notch signaling and its cross-talk pathways.

To develop the model, an intra-cellular molecular reaction network of Notch signaling and its cross-talk pathways are connected to important biomarkers of tumor and non-tumor cells. High-throughput transcriptomics data such as RNA-Seq, microarray and proteomics data such as mass spectrometry, reverse phase protein array, which are generally used for the study of molecular diagnosis and prognosis of cancer cells are used as inputs in the developed models disclosed herein.

Embodiments disclosed herein provide a scoring system to quantify the probability of the grade of tumor in a biological sample which is isolated from a human subject diagnosed with glioblastoma developing low or high grade glioma tumor. The scoring system helps to find out the relative importance of proteins which are responsible for development of glioma tumor cells of different grades and the development of normal brain cells, i.e., neurons and normal astrocytes. This scoring system is based on an “Activity Ratio” (AR) parameter which measures the activities of proteins in different cell types and helps to identify important proteins involved in development of each cell type, i.e. tumor and non-tumor cells.

In embodiments disclosed herein, a method for determining the risk of tumor development grade of Glioblastoma (GBM or Low Grade Glioblastoma) from the general GBM developmental model is provided. The method comprises: collecting tumor samples from patients diagnosed with glioblastoma cancer (101) followed by extracting and purifying whole cell mRNA form the samples (102), performing high-throughput mRNA sequencing process (103) requiring purified and enriched mRNA library for generating the pair-end short reads of the sequences, which is further checked by measuring the library size and base quality and shared in a computer readable text file, processing RNA-Seq files (104) of step (ii) for quality checking of the sequences, aligning of the sequence reads to the human reference genome and quantifying the transcripts/genes, performing differential gene expression analyses (105) to determine the differential mRNA expression profile of the collected tumor specimen with respect to normal cells, producing a predictive computing module (106) based on the tumor specimen, wherein the predictive computing module utilizes the mRNA expression profile comprising select input proteins, performs mechanistic simulations to compute the frequencies of tumorigenic [LGG (Low Grade Glioblastoma) and GBM] and non-tumorigenic cells, followed by computing a novel quantitative (i.e., phenotype predictor) score of each sub-types of glioma tumor cells to predict the chances of occurrence of glioma tumor of respective grades.

In another aspect, the method is based on an activity ratio (AR).

In another aspect, the predictive computing module (106) comprising selecting marker proteins involved in cellular states of phenotypic functions of normal neurogenesis and glioblastoma tumorigenesis in adults are selected from the group consisting of apoptosis, aNSC (Neural Stem Cell) renewal, NPC (Neuron Progenitor Cell) differentiation, ASPC (Astrocyte Progenitor Cell) differentiation, and GBM (Glioblastoma) development. The method comprises defining cellular states, viz. quiescent (or inactive) state, NSC (neural stem cell) renewal, GSC (Glioma Stem Cell) renewal, ASPC (Astrocytes progenitor cell), low and high grade glioblastoma tumor (LGG-I, LGG-II, Grade-IV) states based on activation of phenotypic function and co-expression of multiple marker proteins, (a) simultaneously identifying proteins in the network model that drive the cellular states by measuring the novel activity ratio (AR) scores thereby identifying driver proteins causing tumorigenesis in the subject and classifying the tumors into different grades (Low and High), (b) identifying the risk of a subject developing glioblastoma (low or high) by measuring the novel phenotype predictor score of the cellular states, identifying intra-tumor heterogeneities by assessing the variations of protein expressions with high or low grades of glioblastoma tumor cells and the molecular sub-clones of the tumor cells, screening and identifying a combination of cell signaling proteins as potential drug targets by a novel perturbation analysis and determining the chance of tumor relapse or cell death.

In another aspect, in the method for determining the grade of Glioblastoma (GBM) the predictive computing module (106) comprises; defining a plurality of input, intermediate, and output molecules (e.g., proteins, metabolites) and their intra-cellular biochemical reactions involved in the process of normal neurogenesis and glioblastoma tumorigenesis in adult human brain, selecting marker proteins (or end products of the intra-cellular biochemical reactions) responsible for common phenotypic outcomes observed during normal neurogenesis, writing logical equations for defining the functional and dynamic relationships between the molecules and their connections with the phenotypic expressions, defining a cellular state “Quiescent (inactive)” wherein no phenotypes are observed, randomly assigning the binary initial states (either 1 or 0) to the input proteins and creating 1 million random expression vectors of the input proteins, dividing the 1 million binary expression vectors of the input proteins in 100 separated batches, in which each batch contains 10,000 random expression vectors, starting the simulations for each batch (10,000 initial states) and recording the phenotypic output of each initial state, iterating the process over all 100 batches and simultaneously recording the distributions of the phenotypic outcomes (or cellular states) of each batch, computing the Shannon entropy of the observed phenotypic or cellular outcomes of each simulation batch and selecting the batch with highest entropy value for further analyses to compute the activity ratio (AR) score and frequency distributions of the observed cellular states, checking the cellular state or phenotypes viz. quiescent state, apoptosis, NSC renewal, NPC differentiation, ASPC differentiation, and GSC renewal have arrived in the distribution or not, discarding the results if any of the phenotypes of (x) are missing in the simulation batch and repeating the steps from (iii) to (x) until observing the appropriate distributions of the desired phenotypes or cellular states mentioned in (x) of adult neurogenesis process, selecting the simulation batch with highest entropy if the simulation successfully reproduce the normal and aNSC's developmental process by expressing the phenotypes mentioned in (x) and subsequently compute the activity ratio (−1.44≤AR≤+1.44) of each input protein for individual phenotype or cellular state, validating the activity ratio (AR) score of each protein (i.e., positive AR score for up-regulated/active states=+1.44; negative AR score for down-regulated/inactive states=−1.44) representing each phenotype with the experimental data, such as immunohistochemistry, western blot, mass spectrometry, transcriptomics, etc., repeating the simulation from step (iii) to (xiii), if a protein shows wrong activity in any of the phenotype, otherwise proceed to the next step, (a) declaring the proteins with positive (and maximum=+1.44) and negative (and minimum=−1.44) activity ratio (AR) score observed in a particular phenotype as the driver proteins for the development of that phenotype, for example, the positive and maximum AR score of P53 is the driver of the phenotype apoptosis, naming the simulation strategy from (i) to (xiv) as the model of aNSC development simulation, in which the normal neurogenesis and gliogenesis including natural cell death (apoptosis) processes are successfully simulated and validated with the experimental observations, fitting the distribution of the phenotype prediction scores of each cellular state observed in 100 simulation batches with different distribution functions and calculating the confidence interval of the phenotype predictor score of a cellular state from its corresponding distribution function, finding the cellular state Glioblastoma stem cells (GSC) renewal with input protein P53 at inactive state (i.e., mutation or negative AR score) in the simulation outputs and thus proving the inactivation of tumor suppressor protein P53 as the driver of GSC development from aNSCs in human brain, removing P53 protein from the input protein list made in step (v) and considering the P53 protein as constitutively down-regulated (binary: 0 or False) state in the logical equation model developed in step (iii) to create another new model.

In another aspect, the cellular states are selected from the group consisting of quiescent (or inactive) state, NSC (neural stem cell) renewal, GSC (Glioma Stem Cell) renewal, ASPC (Astrocytes progenitor cell), low and high grade glioblastoma tumor (LGG-I, LGG-II, Grade-IV) states based on activation of phenotypic function and co-expression of multiple marker proteins.

In another aspect, in the method a model with P53 mutation comprises; assessing the observed cellular states and their frequency distributions across 100 simulation batches and identifying the simulation batch with highest Shannon entropy as mentioned in (ix), selecting the simulation batch with highest Shannon entropy for activity ration calculation and assessing the distribution of the frequencies of cellular states, checking whether the cellular state “apoptosis” is present in the frequency distribution or not. If yes, then modify the logical equations as mentioned at step (iii) and repeat the steps from (iii) to (xvi), otherwise proceed to the next step, checking whether the cellular state “GSC renewal” and “ASPC differentiation” are present or not. If not, then modify the logical equations as mentioned at step (iii) and repeat the steps from (iii) to (xvii), otherwise proceed to the next step, calculating the AR score of each input protein driving the cellular state “ASPC differentiation” and extracting those proteins which have maximum & positive (+1.44) and minimum & negative (−1.44) AR scores in the cellular state of “ASPC differentiation”, repeating the simulation from step (iii) to (xix), if a protein shows wrong activity in any of the phenotype, otherwise proceed to the next step, (a) declaring the proteins with positive (and maximum=+1.44) and negative (and minimum=−1.44) activity ratio (AR) score observed in a particular phenotype as the driver proteins for the development of that phenotype, for example, JAK2 and STAT3 proteins showing positive and maximum AR score are the driver of “ASPC differentiation” process and P53 protein having negative AR score=−1.44 is the driver of GSC renewal, renaming the simulation strategy from (xv)-(xix) as the model of “glioblastoma stem cells (GSC) development” model, in which the GSCs show continuous renewal process with no marker sign of apoptosis. GSCs also develop into matured but mutated (P53) astrocytes cells which clearly indicates the development of astrocytoma or glioma, fitting the distribution of the phenotype prediction scores of each cellular state observed in 100 simulation batches with different distribution functions and calculating the confidence interval of the phenotype predictor score of a cellular state from its corresponding distribution function, finding the cellular state Astrocytes progenitor cells (ASPC) differentiation and the AR scores of each protein which have shown either the value of +1.44 or −1.44, removing the proteins with AR score either +1.44 or −1.44 from the input protein list made in step (v) and considering the P53 protein as constitutively down-regulated (binary: 0 or False) state in the logical equation model developed in step (iii) to create another new model.

In another aspect, after determining the risk of tumor development (either LGG or GBM) from the general GBM developmental model by using the patient's mRNA sequencing data, the method of target screening and identifying the potential protein for personalized target-based therapy comprises; extracting the temporal activity profiles of intermediate molecules of the network model from the STG (a State Transition Graph) generated after simulation started from the initial states of the molecules to the cellular state representing high-grade (Grade-V) glioblastoma cellular state; extracting the activity profile of Grade-W glioblastoma cellular state in STG; identifying the delays and correlations of activity profiles of each intermediate molecule with the temporal activity profile observed for high-grade (Grade-V) glioblastoma cellular state by Fast Fourier Transformation (FFT) analyses; extracting the molecules having absolute correlation value ≥0.75 with P value <0.05, and Delay ≤3 with the activity pattern of high-grade (Grade-V) glioblastoma cellular state, and selecting for perturbation analyses; perturbing the molecules by constitutively up-regulating molecules which have negative correlation with high-grade (Grade-V) glioblastoma cellular state in the new perturbation analysis; perturbing the molecules by constitutively down-regulating the molecules which have negative correlation with high-grade (Grade-V) glioblastoma cellular state in the new perturbation analysis; assessing the changes of frequencies of the cellular states representing the low-grade and high-grade glioblastoma cellular states in the perturbation studies with the previous simulation, placing the perturbed molecules at the top of the list on the basis of its ability to maximum suppression of low-grade and high-grade glioblastoma cellular states in the perturbation study, wherein the molecules held rank at the top of the list are considered as potential targets for target-based, personalized glioblastoma therapy.

Embodiments disclosed herein provide a method of implementation of AR score and other methodologies useful for biomarker and drug-targets identification of individual glioblastoma patients in real-life clinical settings. The present application describes the computer-aided implementations of the novel algorithms and methods (computational modules) discussed in real-life clinical settings.

Embodiments disclosed herein also introduce a novel prediction system, which considers transcriptomics or proteomics data of glioma tumor cells as an input to classify tumor cells in the correct histopathological grade. The system also uses a scoring parameter “phenotype prediction score” to quantify the likelihood of the development of glioma tumor.

Embodiments disclosed herein utilize the developed predictive models in a computer implemented method to classify the patient derived glioma tumor samples in different pathological grades and successively predict the patient specific novel combination of drug-targets by analyzing the intra-tumor heterogeneity; said predictive model comprising a plurality of input proteins involved in cellular states of phenotypic functions of normal neurogenesis and glioblastoma tumorigenesis in adults selected from apoptosis (i.e., natural cell death), aNSC (Neural Stem Cell) renewal, NPC (Neuron Progenitor Cell) differentiation, ASPC (Astrocyte Progenitor Cell) differentiation, and GBM (Glioblastoma) development; the method comprising;

(i) defining cellular states, viz. quiescent (or inactive) state, NSC (neural stem cell) renewal, GSC (Glioma Stem Cell) renewal, ASPC (Astrocytes progenitor cell), low and high grade glioblastoma tumor (LGG-I, LGG-II, Grade-IV) states based on activation of phenotypic function and co-expression of multiple marker proteins,

(ii)(a) simultaneously identifying proteins in the network model that drive the cellular states by measuring the novel activity ratio (AR) scores thereby identifying driver proteins causing tumorigenesis in the subject and classifying the tumors into different grades (Low and High),

(ii)(b) identifying the risk of a subject developing glioblastoma (low or high) by measuring the novel phenotype predictor score of the cellular states,

(iii) identifying intra-tumor heterogeneities by assessing the variations of protein expressions with high or low grades of glioblastoma tumor cells and the molecular sub-clones of the tumor cells.

(iv) screening and identifying a combination of cell signaling proteins as potential drug targets by a novel perturbation analysis and determining the chance of tumor relapse or cell death.

It is to be understood that both the foregoing general description and the following detailed description describe various embodiments and are intended to provide an overview or framework for understanding the nature and character of the claimed subject matter. The accompanying drawings are included to provide a further understanding of the various embodiments, and are incorporated into and constitute a part of this specification. The drawings illustrate the various embodiments described herein, and together with the description serve to explain the principles and operations of the claimed subject matter.

### BRIEF DESCRIPTION OF DRAWINGS

FIG. 1 depicts the present protocol for the decision-making steps used in the personalized GBM therapeutics studies;

FIG. 2 depicts the block diagram of the process followed while uploading the RNA-Seq file (BAM/FASTQ) from sequencing machine to high performance computing system for further bioinformatics analyses;

FIG. 3 depicts the workflow of the methods for developing the models useful for detecting the glioma tumor sub-types from patient's mRNA sequencing data;

FIG. 4 depicts the system and methods of implementations proposed to be utilized for computing the risk of glioma tumor of an individual patient;

FIG. 5 depicts methods for identifying novel personalized drug-targets of glioma patients;

FIGS. 6A-6B depict the simulation results depicting Shannon entropy scores (FIG. 6A) and Normalized frequencies of cellular states observed in aNSC, GSC, and GBM models (FIG. 6B). Batch in aNSC, GSC, and GBM models with highest Shannon entropy scores in each model simulations are chosen for calculating normalized frequencies of each attractor states of different models;

FIGS. 7A-7F depict activity profiles of different markers proteins and temporal dynamics of the corresponding fixed-point attractor states observed in aNSC simulation: Quiescent state (FIGS. 7A and 7B); Apoptosis (FIGS. 7C and 7D); NPC Differentiation (FIGS. 7E and 7F);

FIGS. 8A-8J depict activity profiles of different markers proteins and the temporal dynamics of the corresponding cyclic attractor states observed in aNSC simulation: NSC Renewal/NPC Differentiation/Apoptosis (FIGS. 8A and 8B); NSC Renewal/Apoptosis (FIGS. 8C and 8D); NSC Renewal/NPC Differentiation (FIGS. 8E and 8F); GSC Renewal (FIGS. 8G and 8H); ASPC Differentiation (FIGS. 81 and 8J);

FIGS. 9A-9H depict activity profiles of different markers proteins and temporal dynamics of corresponding cyclic attractor states observed in general GBM developmental simulation model: ASPC Differentiation (FIGS. 9A and 9B); GSC Renewal/ASPC Differentiation (FIGS. 9C and 9D); GSC Renewal/ASPC Differentiation/GBM Development (FIGS. 9E and 9F); GSC Renewal/ASPC Differentiation/NPC Differentiation (FIGS. 9G and 9H);

FIGS. 10A-10L depict distributions of the Phenotype Predictor Scores observed for different cellular states in aNSC, GSC, General GBM and Grade-IV GBM models. Distributions are plotted for all distinctly observed cellular states (total 12), namely: Quiescent state (FIG. 10A); Apoptosis (FIG. 10B); NPC Differentiation (FIG. 10C); GSC Renewal (FIG. 10D); NSC Renewal/NPC Differentiation (FIG. 10E); GSC Renewal/NPC Differentiation (FIG. 10F); NSC Renewal/Apoptosis (FIG. 10G); NSC Renewal/NPC Differentiation/Apoptosis (FIG. 10H); GSC Renewal/ASPC Differentiation/NPC Differentiation (FIG. 10I); ASPC Differentiation (LGG-I) (FIG. 10J); GSC Renewal/ASPC Differentiation (LGG-II) (FIG. 10K); and GSC Renewal/ASPC Differentiation/GBM Development (Grade-IV) state (FIG. 10L);

FIGS. 11A-11C depict normalized frequencies observed for LGG-I (FIG. 11A) LGG-II (FIG. 11B) and Grade-IV tumor cells (FIG. 11C) in High-grade GBM model and different target screening scenarios. TC: HIF1A Inhibition; TC2: PI3K & AKT Inhibition; TC3: STAT3 Inhibition; TC4: MASH1 & NGN1 Activation; TC5: PI3K Inhibition & MASH1 & NGN1 Activation; TC6: PI3K & AKT & HIF1A Inhibition (*P-Value ≤0.05);

FIGS. 12A-12C depict Activity Ratio (AR) scores of input molecules of aNSC (FIG. 12A) GSC (FIG. 12B) and GBM Model simulations (FIG. 12C). The AR-scores of each input molecules are calculated for every cellular state. TP53 protein is down-regulated (mutated) in the GSC model simulation. On the other hand, while simulating GBM developmental model, TP53 protein is down-regulated (mutated) along with over-expression of RBPJ+/+, JAK2+/+ and STAT3+/+ proteins;

FIGS. 13A-13D depict comparative statistics of the normalized frequency distributions observed for different cellular states in master aNSC and general GBM models with and without the inputs of TCGA-LGG and TCGA-GBM RNA-Seq expression data.

### DETAILED DESCRIPTION

### Definitions

Normalized frequency of cellular state: The normalized frequency of a cellular state in a particular simulation batch is the ratio of its observed frequency to a total number of random initial conditions.

Shannon entropy: Shannon entropy is used in this application to measure the total information content of a simulation batch by measuring the summation of the negative logarithms of the probability mass functions of all the observed cellular states.

Activity Ratio (AR): Activity Ratio score of any input protein/node with respect to a particular cellular state of the logical model simulation is measured by calculating the total number of times the node is active or inactive within all the random input sequences, which trigger that particular cellular state in the attractor space.

Predictor Score: The predictor score of a particular cellular state observed in the simulation defines as the ratio of the total number of observed occurrences to the total phenotypic cost required for reaching the cellular state in the attractor space.

Phenotypic cost function is a function calculated by measuring the ratio of the average Hamming distance traversed by all the molecules while reaching at the particular phenotype/cellular state to the normalized frequency of the phenotypes.

Hamming distance between two successive nodes (or cellular states) in a State Transition Graph (STG) is the temporal changes or evolution of the expression dynamics of the pathway molecules in successive time points.

In the fixed-point (singleton attractor) state, the binary expressions of the cellular state and the pathway molecules will show homeostatic behavior, whereas in the periodic state it will be oscillatory in nature.

Particular embodiments will now be described in detail in connection with certain preferred and optional embodiments, so that various aspects thereof may be more fully understood and appreciated.

The embodiments disclosed herein narrate the stepwise executions of different computational modules-starting from the transcriptomic data collection from glioblastoma patients to tumor grade prediction, driver gene or biomarker identification, and personalized drug-targets identification.

Embodiments herein disclose a computer implemented process for identifying proteins/genes involved in glioblastoma tumorigenesis, calculating the potential risk of glioblastoma tumor development, classifying the grades of tumor cells, detecting patient specific intra-tumor heterogeneity and tumor sub-clones, and screening personalized potential drug-targets in a subject using mechanistic simulation of a network model.

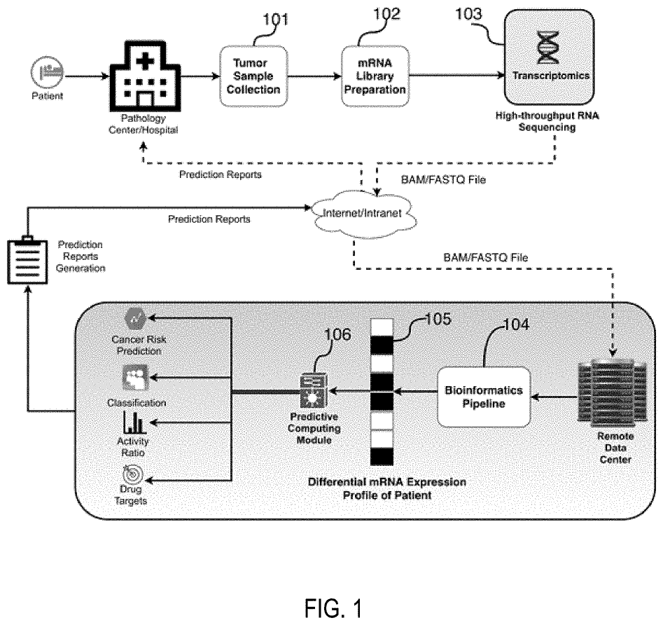

Accordingly, FIG. 1 refers to the stepwise execution protocol of the proposed system, which may be used for prediction of the risk of glioma tumor development, classification of the tumor sample in the respective histopathological grade, detection of driver proteins associated with the development of the tumor grade, and identification of an individual or a combination of potential drug-target(s) to reduce the growth of glioma tumor cells. The protocol described in FIG. 1 may be used when, for example, the high-throughput whole transcriptomics profile (RNA-Seq data) of the tumor sample is available from a brain tumor patient or from an individual who has been diagnosed with a suspicious abnormal growth in the brain tissue after the primary diagnosis performed by the expert.

In a preferred embodiment the illustrations of FIG. 1 provide a computer implemented process for determining the grade of cancer comprising;

(i) collecting tumor samples from patients diagnosed with glioblastoma cancer (101) followed by extracting and purifying whole cell mRNA form the samples (102);

(ii) performing high-throughput mRNA sequencing process (103) requiring purified and enriched mRNA library for generating the pair-end short reads of the sequences, which is further checked by measuring the library size and base quality and shared in a computer readable text file;

(iii) processing RNA-Seq files (104) of step (ii) for quality checking of the sequences, aligning of the sequence reads to the human reference genome and quantifying the transcripts/genes;

(iv) performing differential gene expression analyses (105) to determine the differential mRNA expression profile of the collected tumor specimen with respect to normal cells (control);

(v) producing a predictive computing module (106) based on the tumor specimen, wherein the predictive computing module utilizes the mRNA expression profile comprising select input proteins, performs mechanistic simulations to compute the frequencies of tumorigenic (i.e., LGG and GBM) and non-tumorigenic cells, followed by computing a novel quantitative (i.e., phenotype predictor) score of each sub-types of glioma tumor cells to predict the chances of occurrence of glioma tumor of respective grades.

Accordingly, the tumor sample collection process (101) requires to be performed by an expert surgeon for removing a full or partial segment of the tumor followed by freezing the specimen at low temperatures. The mRNA library preparation process (102) comprises extraction and purification of whole cell mRNA molecules from freshly frozen (FF) tumor specimen and is required to be performed for quantifying and enriching the mRNA library. The high-throughput mRNA sequencing process (103) requires a purified and enriched mRNA library for generating pair-end short reads of sequences, which is further checked by measuring the library size and base quality and shared in either BAM or FASTQ file.

If mRNA library preparation (102) and high-throughput sequencing (103) are not available at tumor resection and specimen collection site (101) or if necrotic and diagnosis are required to be done prior to specimen freezing, the freshly operated tumor specimen maybe fixated in formalin solution. The fixated tumor sample is then embedded in a paraffin wax block for long-term preservation. This formalin-fixed paraffin embedded (FFPE) blocks are used for mRNA extraction, library preparation followed by high-throughput sequencing. The mRNA library and sequencing maybe generated in replicates and if possible same experiments maybe repeated for generating mRNA sequence of the negative control or healthy tissue collected from the same patient. To perform process steps 101, 102, and 103, well-established and highly standardized protocols routinely used for such purposes are to be followed.

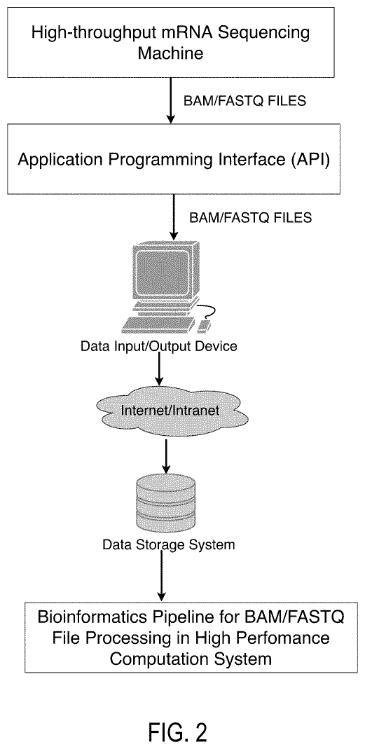

The raw RNA sequence files of all replicates and negative controls generated from the process 103 are required to be stored in a remote data storage system, which is connected with the high-throughput RNA-Seq machine via internet/intranet. FIG. 2 illustrates the block diagram of the entire process, which is followed for uploading the machine generated raw RNA sequence files to the bioinformatics pipeline 104. The RNA-Seq files (FASTQ/BAM) generated from the sequencing machine will be required to transfer in an input/output device, for example a standard desktop computer, by an Application Programming Interface (API) used for connecting the hardware of the sequencing machine with the storage and memory of the I/O device. The device is further connected to the remote data storage system via high-speed internet/intranet connection to transfer voluminous files from the device to the storage system. The sequence files stored in the storage system is required to be easily accessible by the high performance computing machine, for example cluster computers, to further process the large BAM/FASTQ files in the subsequent bioinformatics pipeline (104),

The bioinformatics pipeline (104) maybe used to process RNA-Seq files (both control and tumor specimens) for quality checking of the sequences, alignment of the sequence reads to the human reference genome (e.g., GRCh38), and quantification of the transcripts/genes. State-of-the-art bioinformatics pipelines developed for RNA-Seq reads alignment and quantification processes may be used at this step.

The differential mRNA expression profile (105) of the collected tumor specimen with respect to normal cells (control) is obtained by performing differential gene expression analyses. The count table of all transcripts obtained from each replicate of both groups (i.e., tumor specimen and control cells) shall be used for comparing and identifying the significantly expressed genes (up or down regulation) in tumor cells with respect to control samples. The list of significantly over or under expressed genes identified in the tumor sample is considered as the “transcriptomics profile” of human subject under investigation.

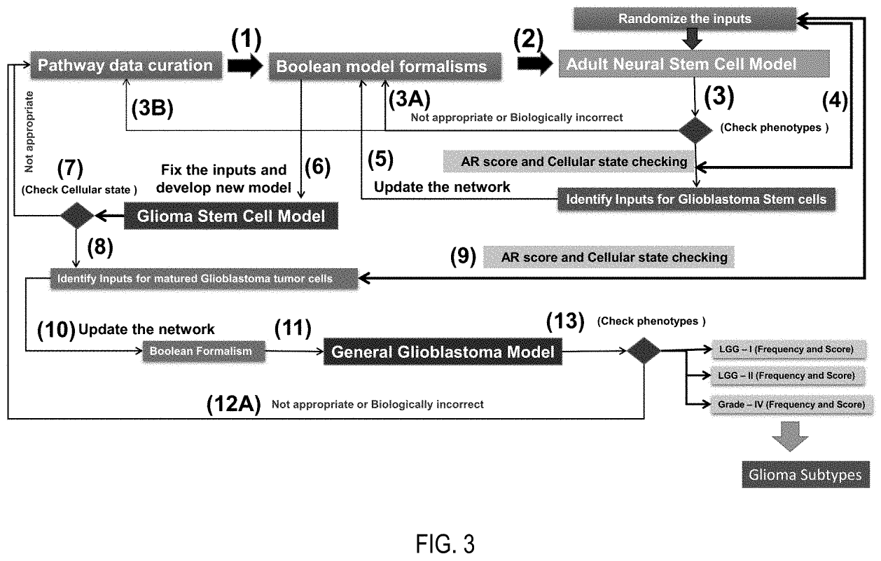

The predictive computing module (106) illustrated in FIG. 1 takes the input of the mRNA expression profile (105) of the tumor specimen of the human subject. The module (106) consists of newly developed pathway models required for performing various tasks of diagnosis and prognosis of glioma or brain tumor. The constructions of the pathway models are performed by step-wise deduction process as illustrated in FIG. 3 comprising:

(i) defining a plurality of input, intermediate, and output molecules (e.g., proteins, metabolites) and their intra-cellular biochemical reactions involved in the process of normal neurogenesis and glioblastoma tumorigenesis in adult human brain,

(ii) selecting marker proteins (or end products of the intra-cellular biochemical reactions) responsible for common phenotypic outcomes observed during normal neurogenesis (viz. Apoptosis, NSC (Neural Stem Cell) renewal, NPC (Neuron Progenitor Cell) differentiation, ASPC (Astrocyte Progenitor Cell) differentiation) and tumorigenesis processes (viz. GSC (Glioblastoma stem cells) renewal, GBM (Glioblastoma) development),

(iii) writing logical equations for defining the functional and dynamic relationships between the molecules and their connections with the phenotypic expressions,

(iv) defining a cellular state “Quiescent (inactive)” in which no phenotypes are observed,

(v) randomly assigning the binary initial states (either 1 or 0) to the input proteins and creating 1 million random expression vectors of the input proteins,

(vi) dividing the 1 million binary expression vectors of the input proteins in 100 separated batches, in which each batch contains 10,000 random expression vectors,

(vii) starting the simulations for each batch (10,000 initial states) and recording the phenotypic output of each initial state,

(viii) iterating the process over all 100 batches and simultaneously recording the distributions of the phenotypic outcomes (or cellular states) of each batch,

(ix) computing the Shannon entropy of the observed phenotypic or cellular outcomes of each simulation batch and selecting the batch with highest entropy value for further analyses to compute the activity ratio (AR) score and frequency distributions of the observed cellular states,

(x) checking the cellular state or phenotypes viz. quiescent state, apoptosis, NSC renewal, NPC differentiation, ASPC differentiation, and GSC renewal have arrived in the distribution or not,

(xi) discarding the results if any of the phenotypes of (x) are missing in the simulation batch and repeating the steps from (iii) to (x) until observing the appropriate distributions of the desired phenotypes or cellular states mentioned in (x) of adult neurogenesis process,

(xii) selecting the simulation batch with highest entropy if the simulation successfully reproduce the normal and aNSC's developmental process by expressing the phenotypes mentioned in (x) and subsequently compute the activity ratio (−1.44≤AR≤+1.44) of each input protein for individual phenotype or cellular state,

(xiii) validating the activity ratio (AR) score of each protein (i.e., positive AR score for up-regulated/active states=+1.44; negative AR score for down-regulated/inactive states=−1.44) representing each phenotype with the experimental data, such as immunohistochemistry, western blot, mass spectrometry, transcriptomics, etc.,

(xiv) repeating the simulation from step (iii) to (xiii), if a protein shows wrong activity in any of the phenotype, otherwise proceed to the next step (xv),

(xiv)(a) declaring the proteins with positive (and maximum=+1.44) and negative (and minimum=−1.44) activity ratio (AR) score observed in a particular phenotype as the driver proteins for the development of that phenotype, for example, the positive and maximum AR score of P53 is the driver of the phenotype apoptosis,

(xiv)(b) naming the simulation strategy from (i) to (xiv) as the model of aNSC development simulation, in which the normal neurogenesis and gliogenesis including natural cell death (apoptosis) processes are successfully simulated and validated with the experimental observations,

(xiv)(c) fitting the distribution of the phenotype prediction scores of each cellular state observed in 100 simulation batches with different distribution functions and calculating the confidence interval of the phenotype predictor score of a cellular state from its corresponding distribution function,

(xiv)(d) finding the cellular state Glioblastoma stem cells (GSC) renewal with input protein P53 at inactive state (i.e., mutation or negative AR score) in the simulation outputs and thus proving the inactivation of tumor suppressor protein P53 as the driver of GSC development from aNSCs in human brain,

(xiv)(e) removing P53 protein from the input protein list made in step (v) and considering the P53 protein as constitutively down-regulated (binary: 0 or False) state in the logical equation model developed in step (iii) to create another new model.

The newly developed model with P53 mutation (constitutively at 0 or OFF state) was simulated by following the same strategy implemented in step (v) to (viii). The stepwise deduction methods (illustrated in FIG. 3) followed to analyze the simulation outputs comprising;

(xv) assessing the observed cellular states and their frequency distributions across 100 simulation batches and identifying the simulation batch with highest Shannon entropy as mentioned in (ix),

(xvi) selecting the simulation batch with highest Shannon entropy for activity ration calculation and assessing the distribution of the frequencies of cellular states,

(xvii) checking whether the cellular state “apoptosis” is present in the frequency distribution or not. If yes, then modify the logical equations as mentioned at step (iii) and repeat the steps from (iii) to (xvi), otherwise proceed to the next step,

(xviii) checking whether the cellular state “GSC renewal” and “ASPC differentiation” are present or not. If not, then modify the logical equations as mentioned at step (iii) and repeat the steps from (iii) to (xvii), otherwise proceed to the next step,

(xix) calculating the AR score of each input protein driving the cellular state “ASPC differentiation” and extracting those proteins which have maximum & positive (+1.44) and minimum & negative (−1.44) AR scores in the cellular state of “ASPC differentiation”,

(xx) repeating the simulation from step (iii) to (xix), if a protein shows wrong activity in any of the phenotype, otherwise proceed to the next step,

(xx)(a) declaring the proteins with positive (and maximum=+1.44) and negative (and minimum=−1.44) activity ratio (AR) score observed in a particular phenotype as the driver proteins for the development of that phenotype, for example, JAK2 and STAT3 proteins showing positive and maximum AR score are the driver of “ASPC differentiation” process and P53 protein having negative AR score=−1.44 is the driver of GSC renewal,

(xx)(b) renaming the simulation strategy from (xv)-(xix) as the model of “glioblastoma stem cells (GSC) development” model, in which the GSCs show continuous renewal process with no marker sign of apoptosis. GSCs also develop into matured but mutated (P53) astrocytes cells which clearly indicates the development of astrocytoma or glioma,

(xx)(c) fitting the distribution of the phenotype prediction scores of each cellular state observed in 100 simulation batches with different distribution functions and calculating the confidence interval of the phenotype predictor score of a cellular state from its corresponding distribution function,

(xx)(d) finding the cellular state Astrocytes progenitor cells (ASPC) differentiation and the AR scores of each protein which have shown either the value of +1.44 or −1.44,

(xx)(e) removing the proteins with AR score either +1.44 or −1.44 from the input protein list made in step (v) and considering the P53 protein as constitutively down-regulated (binary: 0 or False) state in the logical equation model developed in step (iii) to create another new model.

This new development is then simulated by following the same strategy implemented in steps (v) to (viii). However, at this time, the main objective of the simulation is to observe the appearances of low grade glioma (LGG) and high-grade glioblastoma (Grade-IV GBM) cells. The stepwise deduction methods (illustrated in FIG. 3) followed to analyze the simulation outputs comprising:

(i) performing the similar steps from (xv) to (xvii),

(ii) checking whether the cellular state “GSC renewal”, “ASPC differentiation”, “GBM development” are present or not. If not, then modify the logical equations as mentioned at step (iii) and repeat the steps from (iii) to (xvii), otherwise proceed to the next step,

(iii) calculating the AR score of each input protein driving the cellular state “GSC renewal”, “ASPC differentiation”, “GBM development”, and extracting those proteins which have maximum & positive (+1.44) and minimum & negative (−1.44) AR scores in cellular states of “GSC renewal”, “ASPC differentiation”, and “GBM development”,

(iv) repeating the simulation from step (iii) to (xix), if a protein shows wrong activity in any of the phenotype, otherwise proceed to the next step,

(iv)(a) declaring the proteins with positive (and maximum=+1.44) and negative (and minimum=−1.44) activity ratio (AR) score observed in a particular phenotype as the driver proteins for the development of that phenotype,

(iv)(b) renaming the simulation strategy from (xxi) to (xxiii) as the model of “general glioblastoma developmental” model,

(iv)(c) fitting the distribution of the phenotype prediction scores of each cellular state observed in 100 simulation batches with different distribution functions and calculating the confidence interval of the phenotype predictor score of a cellular state from its corresponding distribution function.

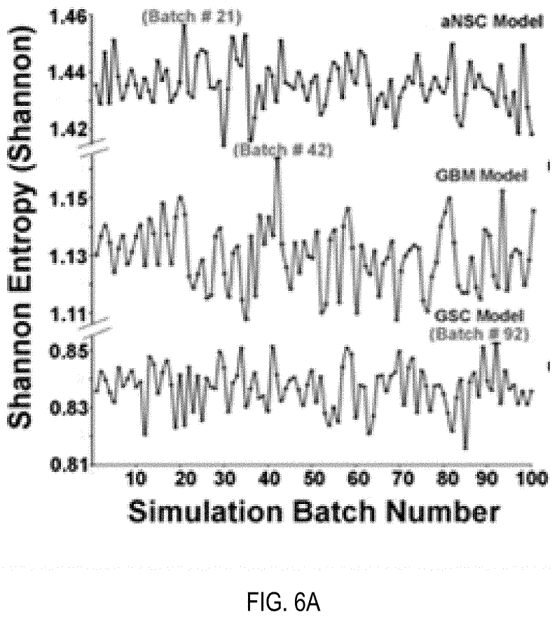

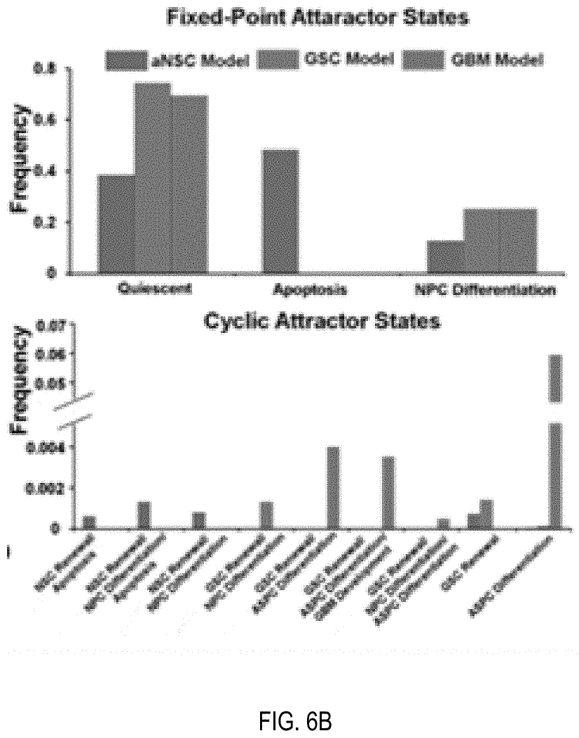

Accordingly, a network model comprising a plurality of input proteins involved in cellular states of phenotypic functions/cellular states were obtained, out of which 3 cellular states, i.e. Quiescent, Apoptosis and Neuron Progenitor Cells (NPC) Differentiation belonged to singleton attractor states referring to either the fully differentiated, quiescent or dead cells (FIG. 6). The remaining 5 cellular states viz. NSC Renewal/NPC Differentiation/Apoptosis, NSC Renewal/NPC Differentiation, NSC Renewal/Apoptosis, Astrocyte Progenitor Cell (ASPC) Differentiation, and Glioblastoma Stem Cell (GSC) Renewal belong to periodic attractor space and mainly correspond to the periodic behavior of cell cycle divisions, i.e. proliferation and growth followed by differentiation or apoptosis. However, unlike differentiation of NPCs which appeared in both the singleton (i.e. NPC differentiation) and periodic (i.e. NSC/NPC, NSC/NPC/Apoptosis) attractor states, differentiation of astrocytes ASPCs were only found in cyclic attractor states. Finally, appearances of these types of cellular states in the steady states level were able to improve the potential of the developed aNSC model to capture the coexistence of heterogeneous cells in the microenvironment of aNSCs, which consists of quiescent, apoptotic, stem-like as well as differentiated neuronal and astrocytes cells.

### Adult Neural Stem Cell Model Development and Simulation

The chemical reactions of the Notch pathway and its cross talks are compiled from previously published model of Notch pathway, which is further modified to simulate the developmental dynamics of aNSCs. The reactions are translated into total of 69 logical equations with 122 logical nodes. The logical nodes consist of 53 input molecules, 39 intermediate molecules, 25 target proteins and 5 phenotypes.

To initiate the Notch pathway activation process, intermediate receptor proteins NOTCH 1/2/3/4 are considered as active and the rest of the molecules (except inputs) are kept as inactive at the initial state. At this stage, the constructed model has no mutation and is simulated for the normal development of aNSCs. The phenotypes are mapped with different marker proteins whose temporal expressions regulate transition dynamics of corresponding phenotypes (Table 1). The simulation outcomes of the model are represented by analyzing the state transition graph (STG), in which the expression vector of all nodes (i.e. molecules) at a particular time is considered as a “state”. The transitions of the states are continued until it reaches the steady-state levels i.e. either at singleton or fixed-point or periodic state.

Two models viz. master aNSC and general GBM are employed in embodiments wherein transcriptomics of glioma tumor patients are used as inputs. At first, the transcriptomics profile is introduced as inputs in the master aNSC model to run a new simulation.

In an embodiment, the aNSCs are highly biased towards development of quiescent, neural progenitor and apoptotic states Analysis of simulation results revealed that normalized frequencies of singleton attractor or cellular states are comparably higher than periodic attractor states (FIG. 6B), and the singleton attractor state “Apoptosis” has highest normalized frequency (Mean 0.494±0.005 S.D.) compared to others cellular states.

Embodiments herein provide a process for determining the risk of future occurrence of GBM tumor of an individual.

Determining Normalized Frequency: The normalized frequencies of each of the cellular states generated from the new simulation are compared with normalized frequencies of the cellular states observed in the master aNSC model. If the observed normalized frequencies are found consistent with respect to the expected normalized frequencies at the significance level (i.e., P-Value ≤0.01), then the inspected subject is categorized as healthy having no risk of developing GBM tumor in the near future. The normalized frequencies are measured by Chi-square goodness-of-fit test. On the other hand, if there is an inconsistency found in the observed and expected normalized frequencies, then the individual's transcriptomics profile would be further tested to detect the risk of developing Low or High grade GBM tumor in future. In this case, the transcriptomics profile was considered as inputs in the master general GBM model and a new simulation will be allowed to run again. If the observed and expected normalized frequencies are well fitted with each other, then it is concluded that the subject has a risk of developing Low-grade glioblastoma (LGG) in future.

Determining Predictor score: To quantify the chances of occurrences of tumor cells of the subject, the phenotype predictor scores are determined for each tumorigenic state i.e. LGG-I, LGG-II and Grade-IV glioblastoma, which will be further helpful to assess the severity of tumor progression.

If it is observed that the expected and observed normalized frequencies are not consistent with each other and the phenotype predictor score of all the tumorigenic states is increased many folds as compared to the master general GBM model, then it is concluded that the subject has a risk of developing high grade GBM tumor. Quantification and further assessments of the chances of developing high-grade GBM tumor can be calculated by measuring the corresponding values of the phenotype predictor scores. Followed by these analyses, for high-grade GBM tumor patients, another simulation will be performed by using the omics profile as input in the master Grade-IV GBM model to screen and rank suitable drug targets for personalized, target based GBM tumor therapy.

In another embodiment, the activity ratio score is determined in step (ii)(a) by determining the contribution of an arbitrary pathway molecule (Xᵢ) in the cell signaling network to drive cellular dynamics towards a particular cellular state. Accordingly, the calculation of activity ratio score comprises the following steps:

(i) identifying the total number of input proteins (I), which do not have any upstream activator and inhibitor in the network model,

(ii) identifying the expressions of the proteins/genes (G) in a subject through proteomics or transcriptomics (microarray, RNA-Seq etc.) analyses, where G⊆I,

(iii) identifying a sub-set of input proteins whose expressions are not determined experimentally, i.e. I−G=X,

(iv) generating a set of N number of expression vectors of total X number proteins, each consisting of randomly chosen binary element Xᵢ={0,1},

(v) simulating the network model by considering input vectors sequentially and detecting the cellular states C={C¹, C², C³, . . . , C^{m}},

(vi) identifying the expression exponent (γ)_{X}_{ᵢ}_{∈X|C}_{^{j}}_{∈C} of an arbitrary protein (Xᵢ) in any cellular state C^{j}, which has appeared in total |S| number of simulation instances out of total N number of simulations by the following equation (5),

$${( γ )}_{{X}_{i} ∈ X \vert {C}^{j} ∈ C} = \frac{1}{\vert S \vert} [ {\sum }_{l = 1}^{\vert S \vert} ( {Y}_{l} \text{:} {X}_{i} = 1 ) - {\sum }_{l = 1}^{\vert S \vert} ( {Y}_{l} \text{:} {X}_{i} = 0 ) ] \qquad ( 5 )$$

Wherein, C=Set of Cellular States={C¹, C², C³, . . . , C^{m}}
m=Total Number Phenotypes observed
Xᵢ∈X=Set of Input Molecules={X₁, X₂, X₃, . . . , X_{X}}
X=Total Input proteins
Y:=Set of Input sequences={Y₁, Y₂, Y₃, . . . , Y_{|S}_{ₖ}_{|}} reaching to cellular state C^{j}
Where, |S|=Total number of input sequences reaching to a particular cellular states out of N random sequences i.e. |S|⊆N

(vii) calculating the activity ratio of an arbitrary input molecule (Xᵢ) in the development of a cellular state C^{j} by the following equation 4:

$$Activity Ratio = {( AR )}_{{X}_{i} ∈ X \vert {C}^{j} ∈ C} = {{Log}_{2} ( {e}^{{γ}_{{X}_{i}}} )}_{{X}_{i} ∈ X \vert {C}^{j} ∈ C} \qquad ( 4 )$$

If AR score of a protein is either +1.44 or −1.44 in a given cellular state, then it can be said that the specific protein is highly essential and positive or negative inducer, respectively for that particular cellular state. This scoring technique is specifically useful for assessing the biological activity of a protein in the development of different grades of tumor cells during tumorigenesis.

In yet another embodiment, the a predictor score is provided in step (ii) (b), wherein the score is measured as the ratio of the total number of observed occurrences (P_{C}_{ᵢ}) to the total cost (Ψ_{C}_{ᵢ}) requires for reaching at it cellular state (Cᵢ) in the attractor space. Predictor score (Cᵢ) is calculated for each cellular state (Cᵢ) and higher its value, greater the chance of reaching to the particular cellular state (Cᵢ). Calculation of the predictor score comprises the following steps:

(i) extracting the state transition graph (STG) of the logical model simulation, wherein the expression states of all the molecules of network model at any arbitrary transition time Zᵢ={Mₜ¹, Mₜ², Mₜ³, . . . Mₜ^{m}:m=Total molecules} is considered as “node”. Node Zₜ is connected with the next transition state Zₜ₊₁, where Z₀ is the initial state (either Up or Down regulation) of all the molecules at time t=0,
Initial states of the molecules Z₀ is considered from the subject's/patient's proteomics/transcriptomics profile.

(ii) identifying the steady state solutions (either fixed-point or cyclic) of the transition nodes {Zₜ, Zₜ₊₁, Zₜ₊₁, . . . Z_{t+T}:T=Total simulation time} obtained in the STG by using the following conditions,
iff Zₜ=Zₜ₊₁=Sₜ=Fixed-point attractor
   Zₜ=Zₜ₊ₛ=Lₜ=Cyclic attractor

(iii) mapping the steady state points with the cellular states by measuring the activities of the marker proteins corresponding to the cellular states. If all marker proteins of a said cellular state are found active in the steady state, then that steady state would be denoted with that cellular state,

(iv) calculating the average state transition cost Φ_{C}_{ᵢ} of all the transitions starting from initial state to a particular steady state (or a cellular state) in the STG by using the following equation. This parameter denotes the average number of molecular changes required during cell signaling activity to develop a particular cellular state (Cᵢ) (e.g. tumor cell) in developing/differentiating stem cells,

$$\begin{matrix}{Φ}_{{C}_{i}} = \frac{ϕ ( {C}_{i} )}{{P}_{{C}_{i}}} & ( 6 ) \\ Where , ϕ ( {C}_{i} ) = \frac{{\sum }_{t = 0}^{s} {d}_{{S}_{t} \to {S}_{r + 1}}}{s} + \frac{{\sum }_{t = 0}^{l} {d}_{{L}_{t} \to {L}_{t + 1}}}{l} & ( 7 )\end{matrix}$$

Where, s=Total number of transition states require to reach at the steady state;

$$l = 1 for Singleton attractor \& {\sum }_{t = 0}^{l} {d}_{{L}_{t} \to {L}_{t + 1}} = 0$$

>1 for Periodic attractor (=Periodicities of the cyclic attractor)
μ=Total mutational costs of the entire model
d_{S}_{ₜ}_{→S}_{ₜ₊₁} & d_{L}_{ₜ}_{→L}_{ₜ₊₁}=Hamming distance between two states in the transient and periodic paths
P_{C}_{ᵢ}=Total number of occurrences of Cᵢ in a simulation batch

(v) calculating the total mutation cost Ω induced in the developing/differentiating stem cells within a subject due to the genetic mutation by using the following equation (9),

$$Ω = μ \times \frac{\vert {C}^{n} - ( {C}^{n} ⋂ {C}^{m} ) \vert + \vert {C}^{m} - ( {C}^{n} ⋂ {C}^{m} ) \vert}{( \vert {C}^{n} ⋃ {C}^{m} \vert )} \qquad ( 9 )$$

### Wherein,

(Cⁿ:C₁ⁿ, C₂ⁿ, C₃ⁿ, . . . C_{X}ⁿ)=Cellular states observed without any mutation
(C^{m}:C₁^{m}, C₂^{m}, C₃^{m}, . . . C_{X}^{m})=Cellular states observed with mutation
μ=Total number of genetic mutations carried by the subject/patient
|Cⁿ∪C^{m}|=Total number of cellular states
|Cⁿ−(Cⁿ∩C^{m})|=Total number of cellular states which are not appeared after inducing mutation
|C^{m}−(Cⁿ∩C^{m})|=Total number of cellular states which are newly appeared after inducing mutation

(vi) calculating the total phenotypic cost (Ψ_{C}_{ᵢ}) to reach a particular cellular state (Cᵢ) (e.g. tumor state) in the STG according to the following equation (10);

Ψ_{C}_{ᵢ}=Φ_{C}_{ᵢ}+Ω (10)

(vii) calculating the predictor score[Θ(Cᵢ)] of a particular cellular state (Cᵢ) by measuring the ratio of the total number of observed occurrences (P_{C}_{ᵢ}) of that cellular state (Cᵢ) in the STG to the total cost (Ψ_{C}_{ᵢ}) requires for reaching at that cellular state (Cᵢ). The equation is as follows.

$$Θ ( {C}_{i} ) = \frac{E ( {P}_{{C}_{i}} )}{E ( {Ψ}_{{C}_{i}} )} \qquad ( 11 )$$

Where, E(P_{C}_{ᵢ}) & E(Ψ_{C}_{ᵢ})=Expected values of P_{C}_{ᵢ} and Ψ_{C}_{ᵢ}

The predictor score is particularly useful for predicting and quantifying the chances of developing a specific grade of glioblastoma tumor of a subject/patient in the future.

Further, the phenotypic cost calculated in step (VI) is a function calculated by measuring the ratio of the average Hamming distance traversed by all the molecules while reaching at the particular phenotype/cellular state to the normalized frequency of the phenotypes.

In one more preferred embodiment, target screening is performed by identifying the intermediate molecules of network model, which are mutually interconnected and highly correlated with the temporal activity pattern observed for high-grade (Grade-IV) glioblastoma tumorigenic cellular state.

### Implementation of Phenotype Prediction Score in Tumor Risk Prediction

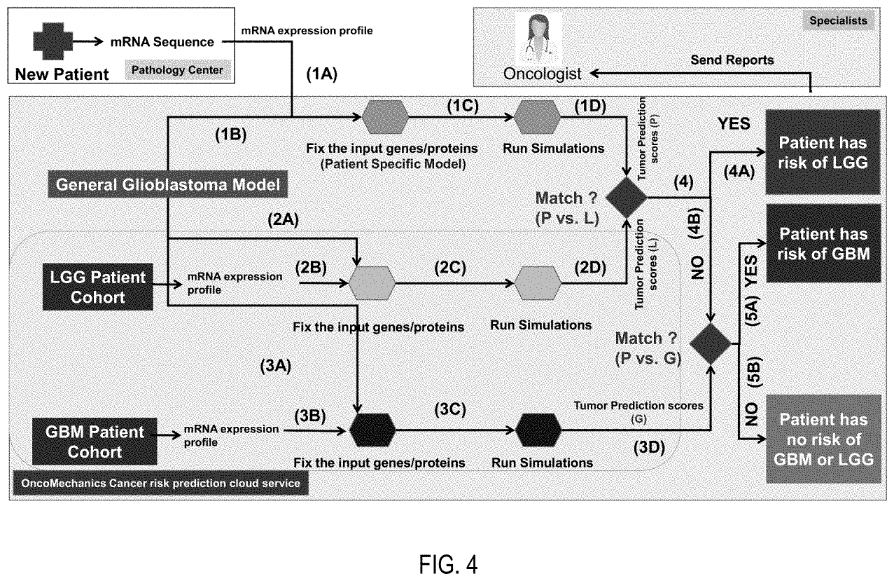

The method for determining the risk of the development of glioma of a new patient is depicted in FIG. 4. The mRNA expression profile generated from the patient will be taken as input to the general GBM developmental model followed by calculation of phenotype (tumor) prediction score (P) described in FIG. 3. The pre-calculated prediction score of low grade glioma computed from LGG patient cohort (L) and the high-grade glioma (G) from the GBM patient cohort (described in FIG. 3) will be stored in the system's backend. For individual patients, the distribution of phenotype distribution score of patient (P) will be compared and fitted with (L). Chi-square goodness of fit test is utilized for this purpose, based on null hypothesis that there is no significant variance in the distribution of P and L. If the significant difference (P≤0.01) is observed then null hypothesis will be rejected and it will be compared with phenotype prediction score (G) observed from the high-grade GBM patient cohort. The significant level of difference will be measured here, and if the null hypothesis (i.e., there is no variation between P and G) is rejected, and patient will be declared as risk free of either GBM/LGG. If P and L are equal, and if there is no significant difference between the two distributions then the patient will be declared to have a risk of developing low grade glioma (LGG). In case if no significant difference is observed between P and G, the patient will be declared to have the risk of developing high-grade GBM (Grade-IV).

### Implementation of Personalized Drug-Target Screening

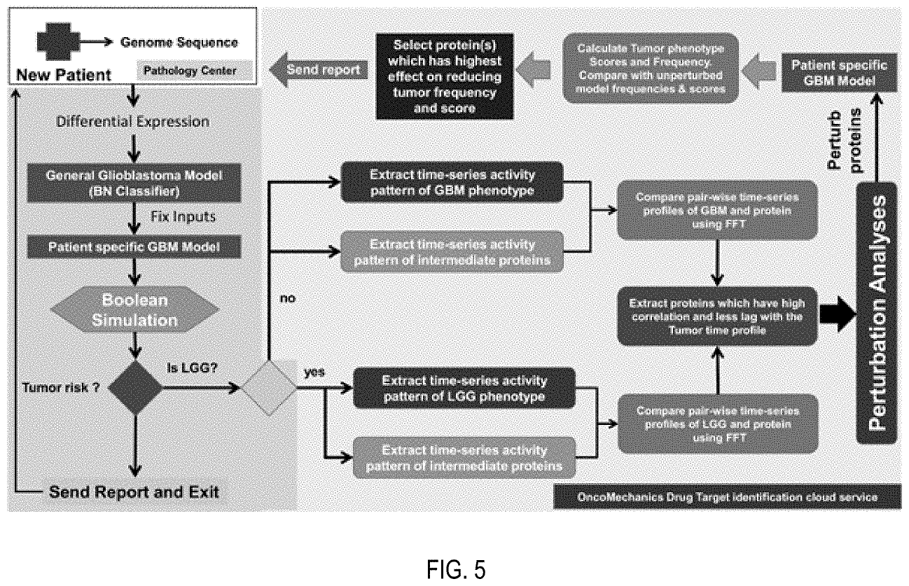

The method for determining the personalized drug-targets for individual patient is illustrated in FIG. 5. After determining the risk of tumor development (either LGG or GBM) from the general GBM developmental model by using the patient's mRNA sequencing data, the method of target screening and identifying the potential protein for personalized target-based therapy comprises:

(i) extracting the temporal activity profiles of intermediate molecules of the network model from the STG generated after simulation started from the initial states of the molecules to the cellular state representing high-grade (Grade-IV) glioblastoma cellular state;

(ii) extracting the activity profile of Grade-IV glioblastoma cellular state in STG;

(iii) identifying the delays and correlations of activity profiles of each intermediate molecule with the temporal activity profile observed for high-grade (Grade-IV) glioblastoma cellular state by Fast Fourier Transformation (FFT) analyses;

(iv) extracting the molecules having absolute correlation value ≥0.75 with P value <0.05, and Delay ≤3 with the activity pattern of high-grade (Grade-IV) glioblastoma cellular state, and selecting for perturbation analyses;

(v) perturbing the molecules by constitutively up-regulating molecules which have negative correlation with high-grade (Grade-IV) glioblastoma cellular state in the new perturbation analysis;

(vi) perturbing the molecules by constitutively down-regulating the molecules which have negative correlation with high-grade (Grade-IV) glioblastoma cellular state in the new perturbation analysis;

(vii) assessing the changes of frequencies of the cellular states representing the low-grade and high-grade glioblastoma cellular states in the perturbation studies with the previous simulation.

(viii) placing the perturbed molecules at the top of the list on the basis of its ability to maximum suppression of low-grade and high-grade glioblastoma cellular states in the perturbation study. The molecules held rank at the top of the list are considered as potential targets for target-based, personalized glioblastoma therapy.

The similar strategies from steps (i) to (viii) may be utilized for identifying drug-targets for LGG patients.

RNA-Seq Data Analyses and Differential mRNA Expression Study

The raw HTSeq counts data are further processed and analyzed for differential gene expression (DEG) analysis to find out the transcripts, which are significantly expressed in the LGG and GBM tumor cells in comparison to the normal, solid tumor cells.

### Drug Targets Screening and Ranking

The molecules, which are significantly positive and negative inducer of high-grade (Grade-IV) cellular state, are extracted through FFT analyses performed on the time course activity sequences of all the intermediate proteins of Notch signaling network. The correlation and delay of the trajectories of the temporal expression dynamics of all the molecules are measured with respect to the reference trajectory of Grade-IV cellular state observed in the STG of “Grade-IV GBM model” simulation study.

In another embodiment, activity ratio scores are provided, indicating proteins selected from the group comprising EP300, RBPJ, APH1A, NCSTN, PSENEN, SNW1, HAT1, and MAML1 are highly expressed (i.e. having maximum AR scores) in all the cyclic attractor states. These said proteins are the core components of Notch pathway, which maintain the balance between self-renewal and differentiations of aNSCs by helping transcriptions of the target genes (HES 1-7/HEY 1, 2, L).

In yet another embodiment, protein molecules showing maximum delay ≤3 and correlation ≥0.6 with the target signal are selected to screen the suitable proteins as drug targets (Table 5).

In another embodiment, the application of the Boolean network model is improvised to simulate the human neural/glioma stem cell development, self-renewal, differentiation, and apoptosis processes.

It considers numerous combinations of binary input states (i.e., up or down-regulation) of the cell signaling molecules of Notch and its cross-talks pathways to observe the possible phenotypic conditions of the neural and glioma stem cells at the steady-states. Although Boolean models were previously used for simulating the biological systems, its implication in the prediction of the risk of developing histopathological grades of glioma is newly proposed in the embodiments of this disclosure. Additionally, the use of Fast Fourier Transformation (FFT) on the state transition dynamics of the tumorigenesis models to predict the possible vulnerable points in the network structure is newly introduced in embodiments herein, which finally helped to identify the important drug-targets in the glioblastoma tumor.

### EXAMPLES

The following examples are given by way of illustration only, such that the person having ordinary skill in the art would recognize that the examples are not to be construed to limit the scope of this disclosure or the claims in any manner.

### Example 1

### Construction of the Logical Model

Notch signaling pathway and its cross-talk reactions with other pathway molecules were considered to construct a logical dynamic model to understand the neurogenesis of adult neural stem cells (aNSCs) in the SVZ of the human brain. Preliminary data for constructing the core pathway model was taken from the logical dynamic model of the Notch pathway by Chowdhury & Sarkar (2013) Clin Exp Pharmacol 3(137):2161-1459. Based on this pathway data, the entire model was then modified and restructured to simulate developmental dynamics of aNSCs and mutated Glioblastoma stem cells (GSCs). The overall reaction mechanisms of this pathway are translated into logical equations using universal logic gates AND, OR and NOT. It was observed that to reach a particular cellular phenotype, a specific set of molecules in the signaling cascade get activated, which forms a functional module or a sub-network and helps to express a class of marker proteins responsible for a specific cell type. Eq. 1-2 were considered while constructing the dynamic Boolean model of Notch along with its cross talk reactions. The initial states (1/ON or 0/OFF) of the input nodes were chosen randomly by generating a random number for each input node from uniform distribution (Unif(0,1)) in the range of 0 to 1 and setting the cut-off at 0.5 (Eq. 2).

$$\begin{matrix}{X}_{i} ( t + 1 ) = {X}_{i} ( 0 ) iff ∀ {x}_{i} ∈ Input Nodes = {f}_{i} ( {X}_{j}^{1} ( t ) , {X}_{j}^{2} ( t ) , {X}_{j}^{3} ( t ) , … , {X}_{j}^{{k}_{i}} ( t ) ) iff ∀ {x}_{i} ∈ Intermediate and Output Nodes & ( 1 ) \\ \begin{matrix} With , initial conditions : {X}_{i} ( 0 ) = 1 iff Unif ( 0 , 1 ) \geq 0.5 \\ = 0 otherwise\end{matrix} & ( 2 )\end{matrix}$$

Where, kᵢ=Total number of Input Nodes

The pathway consists of 117 molecules out of which 53 were input molecules that refer to molecules that do not possess any upstream regulators and during the signaling event they can be at either up-regulated (ON) or down-regulated (OFF) state (Eq. 1). Hence, the possible highest numbers of expression patterns of these input molecules are 2⁵³. In presence of such enormous possibilities, mainly occurring due to variations in expressions of several extrinsic and intrinsic molecules, the adult and inactive NSCs in the neurogenic niche of SVZ have to make a right decision to opt for any of the cellular phenotypes either by proliferation and differentiation during the developmental process or also choose to stop its cell division or undergo apoptosis.

To capture such huge possibilities at equilibrium, a robust simulation technique to stimulate the developmental dynamics of aNSCs with all input states was required. However, logical dynamic simulations by considering all input conditions and finding all possible attractors at equilibrium state are not feasible in terms of required computational cost. Hence, to reduce the computational cost, a random fraction, non-redundant initial input sequences were generated by using uniform random number distribution and subsequently assigned binary states ON (1) or OFF (0) to each input node of the reconstructed model (Eq. 2). The entire random input sequences (10⁶) are then further divided into 100 separate simulation batches (10,000 random sequences) and simulations are performed for all the 100 batches.

### Example 2

### Marker Proteins of aNSC, GSC and GBM Models

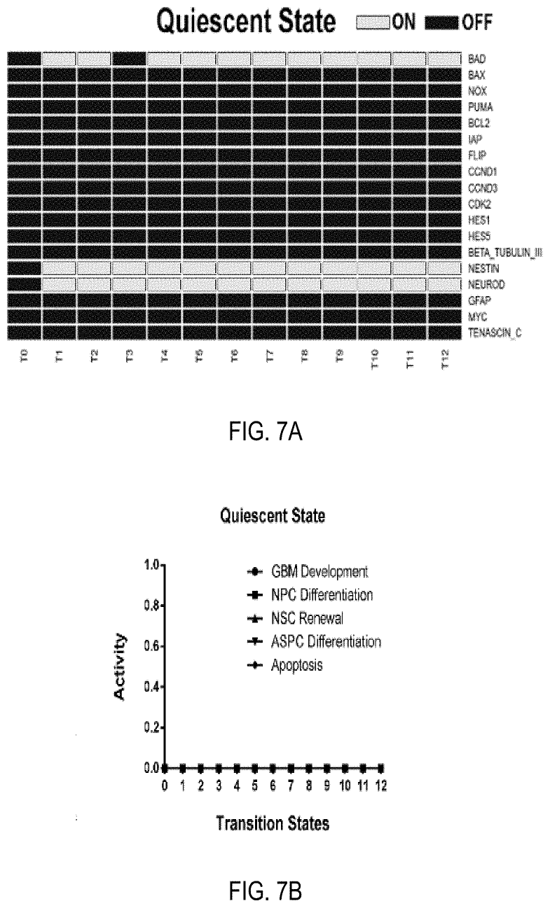

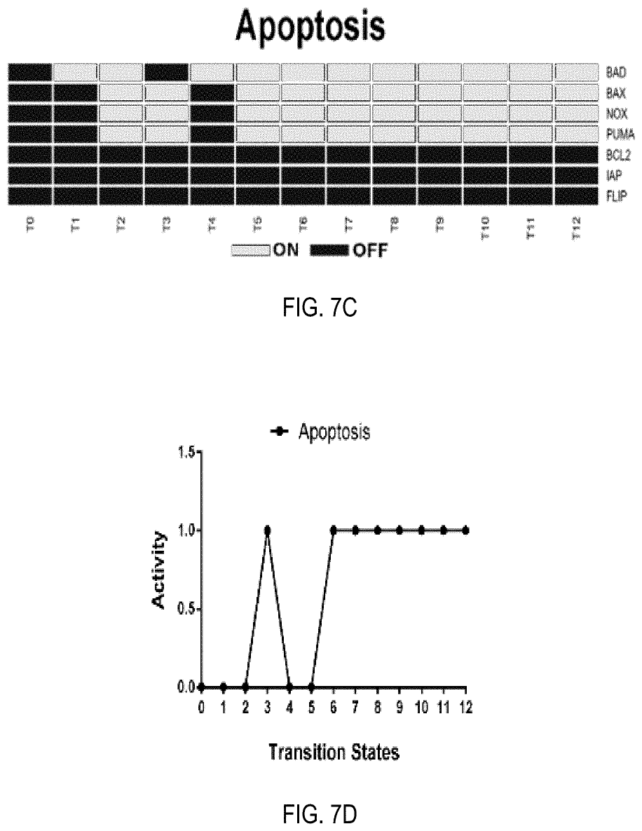

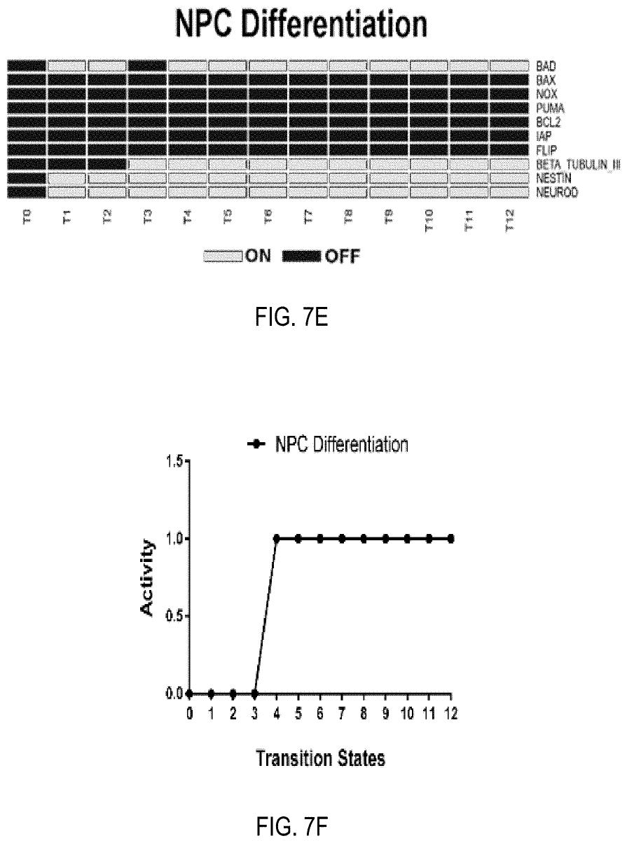

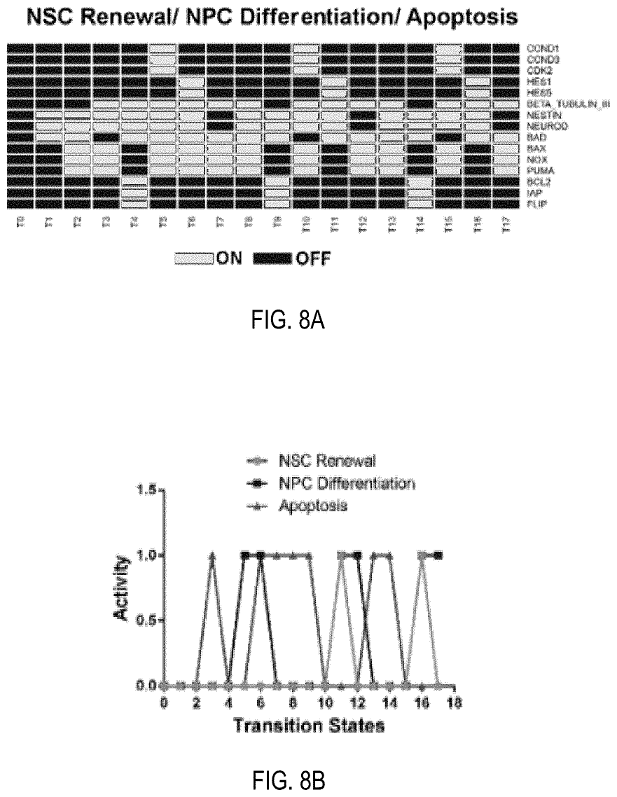

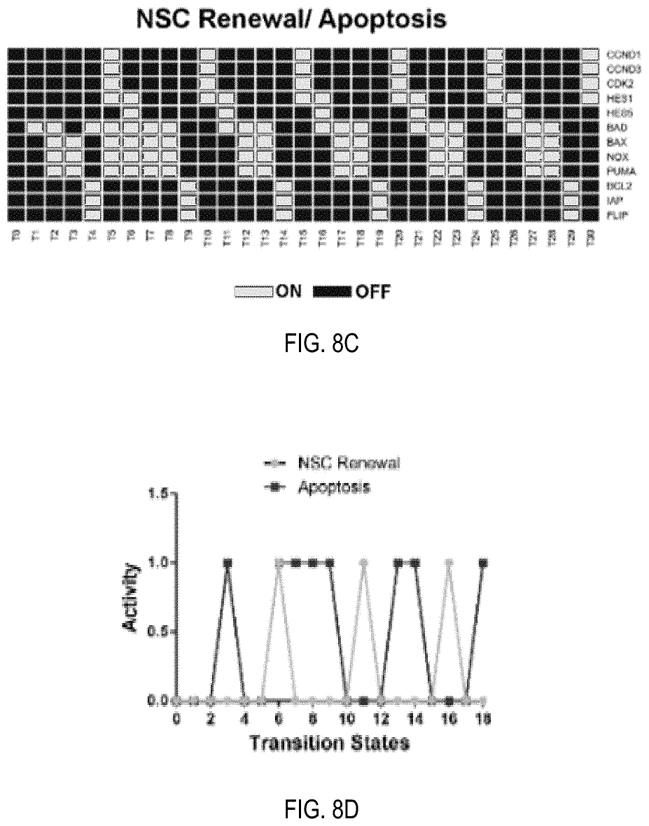

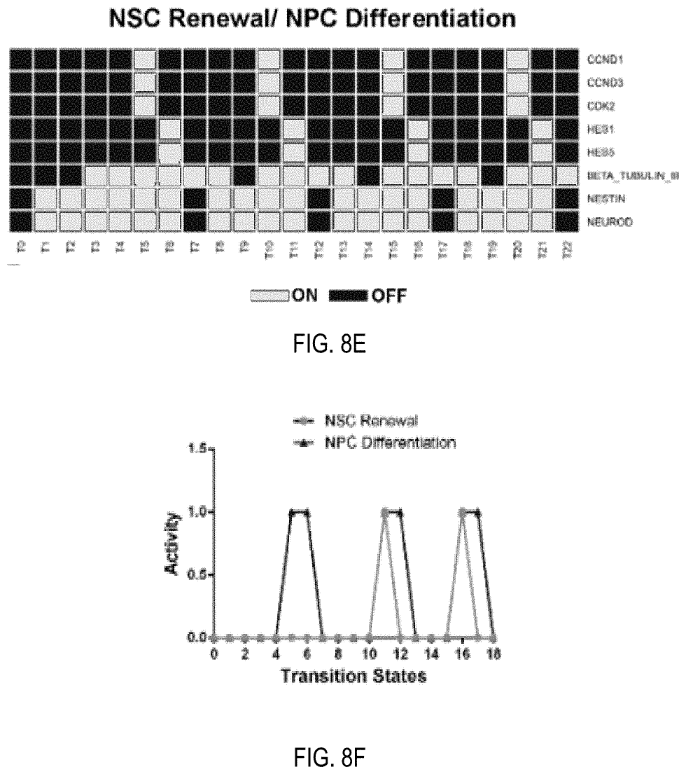

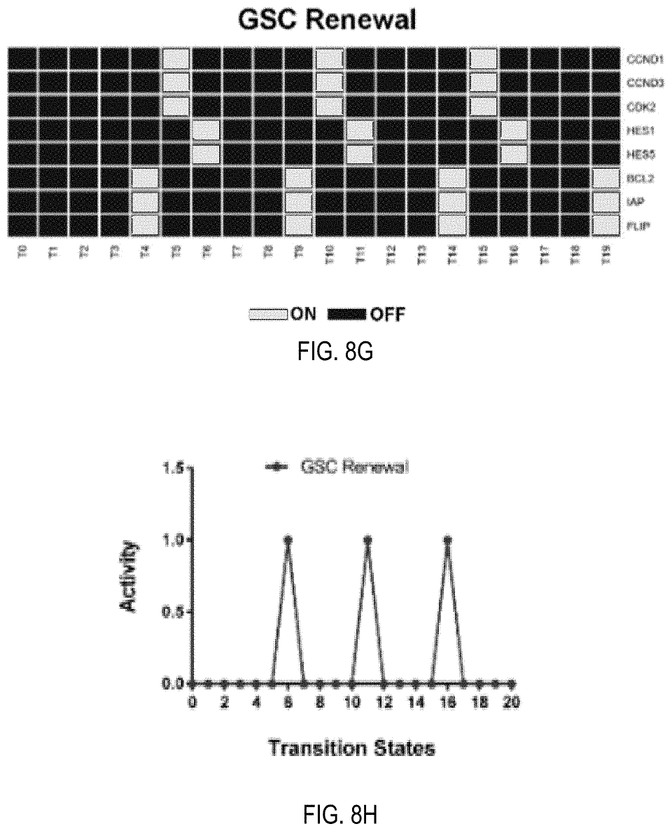

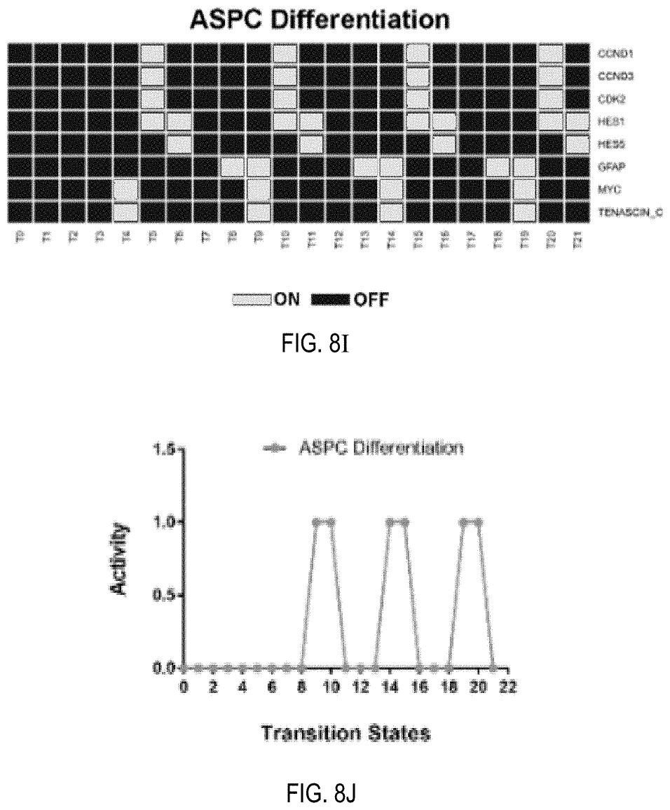

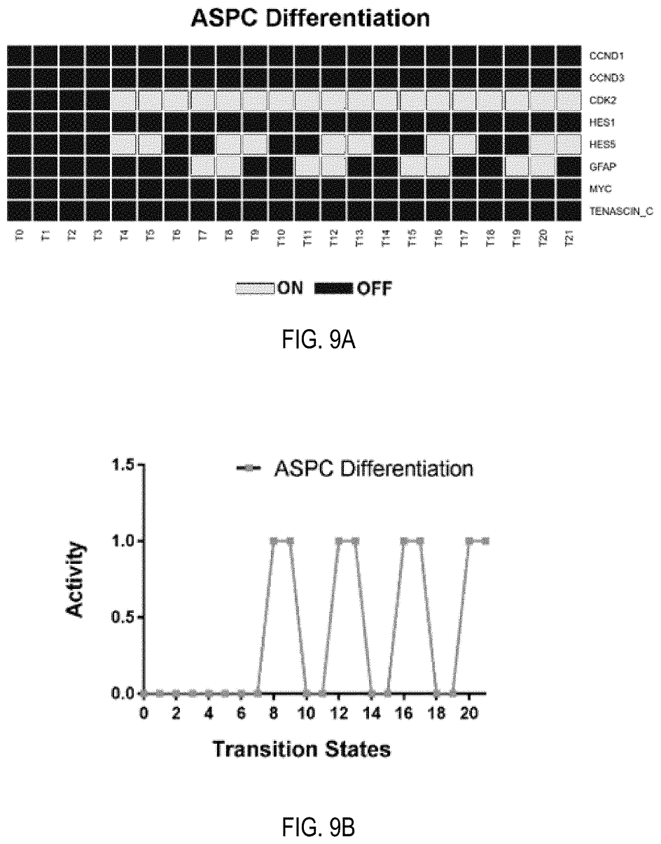

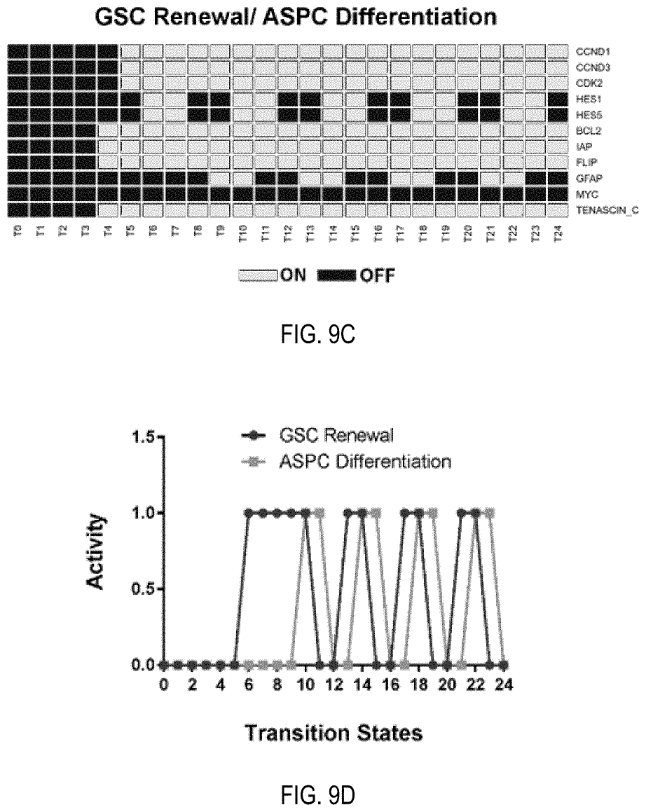

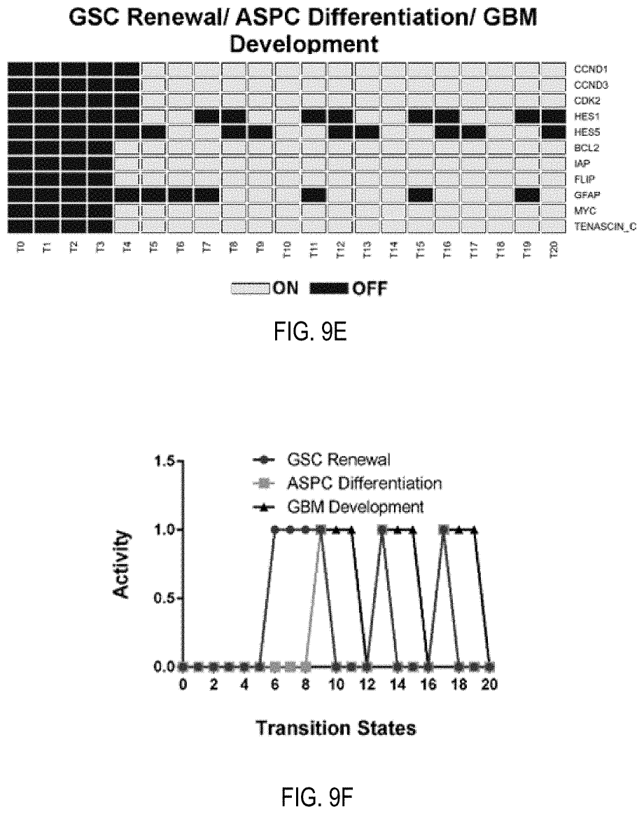

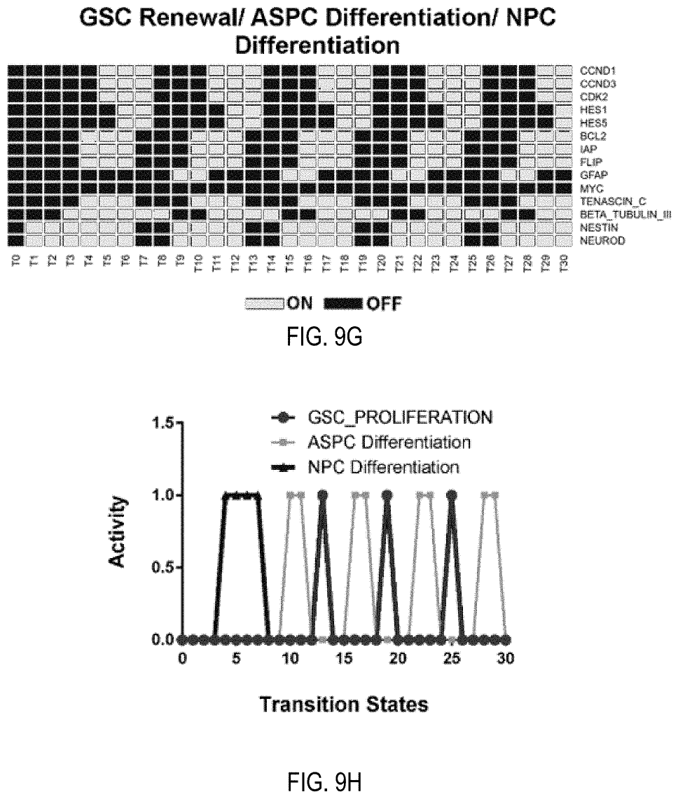

The marker proteins whose expressions are analyzed to observe dynamics of different cellular states or phenotypes in the aNSC, GSC and GBM models are enlisted in Table 1. Oscillatory behaviors of the state transitions of marker proteins were observed in the corresponding cyclic attractor states (e.g. NSC Differentiation), whereas steady state saturation (high or low) was observed for marker proteins in fixed-point attractor states (FIG. 7, FIG. 8, FIG. 9).

TABLE 1
Marker proteins mapped with different phenotypes

| Cellular Phenotypes | Marker Proteins |
|---|---|
| Apoptosis | Pro-apoptotic: PUMA, NOX, |
| | BAD, BAX |
| | Anti-apoptotic: FLIP, IAP, |
| | BCL2 |
| Neural Stem Cells Renewal | CYCLIN-D3, CYCLIN-D1, CDK, |
| | HES1, HES5 |
| Neural Progenitor Cells | NESTIN, NEUROD, β-TUBULIN-III |
| Astrocyte Progenitor Cells | GFAP |
| Glioblastoma Stem Cells | CYCLIN-D3, CYCLIN-D1, CDK, |
| | HES1, HES5, FLIP, IAP, BCL2 |
| Glioblastoma tumor Cells | CYCLIN-D3, CYCLIN-D1, CDK, |
| | C-MYC, TENASCIN-C, GFAP |

The expressions of these marker proteins are denoted in the model simulation by discrete binary states 0 or 1. The occurrences of a particular phenotypic state in the model are mapped with a Boolean function of the corresponding marker proteins of that phenotype and therefore its expression values will be varied in the discrete binary domain of {0,1}.

ƒ: M→P

ƒ:=Boolean Function

P:=Phenotypes ∈{p₁, p₂, p₃, . . . }∈{0,1}

M:=Marker Proteins ∈{m₁, m₂, m₃ . . . }∈{0,1}

### Example 3

### Determining Phenotypes and Cellular States

A total of five phenotypic functions were considered in the developed dynamic model, which are dependent on expression of specific marker proteins of normal and tumorigenic brain cells. These phenotypes are i) Apoptosis, ii) NSC Renewal, iii) NPC Differentiation, iv) ASPC Differentiation, and v) GBM Development. Depending on the distribution of input signals different marker proteins were expressed with different distribution, which in turn regulate expressions of different phenotype in the binary domain {1,0}. Theoretically, it can be considered that in total 32 single and combinations of phenotypes are possible to occur with equal probability in the attractor distribution space. The probability to reach any of the phenotypes would be unbiased if the logical model is purely random. However, in reality, the topology of the constructed logical model is not completely random and unbiased in nature as logical rules defined for Notch signaling network is taken from experimental evidences.

The normal functioning of Notch signaling network is specifically oriented towards neural stem cell renewal and its differentiation into neural progenitor cells. It was also observed that at the time of Notch pathway simulation, only a few phenotypic states occur in attractor space and those states are denoted as cellular states. A cellular state can be an individual phenotype or a combination of multiple phenotypes. The following attributes were considered during model simulation to define two typical cellular states: Quiescent cells and GSC Renewal.
Cellular States:=Quiescent(t)=1 iff ∀ Phenotypes: Pᵢ(t)∈{0}; where, i=1, 2, . . . , 5 GSC Renewal(t)=NSC Renewal(t) and not Apoptosis(t)

Quiescent state is a distinct cellular state found at ON state only when all phenotypes are at OFF state. In this state all other cellular activities such as proliferation, differentiation and apoptosis are found at the dormant stage or at a lower rate. GSC renewal will be at ON state if the phenotype Apoptosis is at OFF state and NSC Renewal is at ON state. It was also observed that based on steady state distributions and temporal expressions patterns (1 or 0) of intermediate and target molecules of Notch pathway, these cellular states were found in either “Fixed-point” or “Periodic” attractors space.

### Example 4

### Calculation of Normalized Frequencies of Cellular States, Shannon Entropy and Activity Ratio (AR) Scores

To reduce the computational cost and time to find out all possible attractor states, a simulation based search algorithm was used to find the maximum number of attractors of the present logical model. The basic criterion of this algorithm was to run the simulation by taking randomly non-redundant, finite set of initial states (N=10⁶) from the overall population (9.0071993×10¹⁵) of input states. The random initial states were further divided into 100 separate batches (i.e. each batch will contain 10,000 random input sequences) and frequencies of all attractor states (cellular states) are calculated for each batch of simulation (FIG. 6A). The frequencies of each cellular state were further normalized by dividing its total frequency observed in a simulation batch with respect to the total number of random initial conditions used for simulation. It should be noted that degeneracy of the observed attractor states with respect to a particular cellular state is possible, which means multiple sets of similar attractor states can be mapped with a particular cellular state depending on the expression state(s) of the marker protein(s) in those attractor distribution Π(A). Attractor distributions are calculated from initial input states distribution Π(I) in any simulation batch. Hence, distribution of cellular states Π(C) is also dependent on distribution of attractor states Π(A) and can be mapped with Boolean function ƒ: Π(A)→Π(C).

In an arbitrary batch of simulation (Bᵢ), there exists a set of cellular states Cᵢ={Cᵢ¹, Cᵢ², Cᵢ³, . . . , Cᵢ^{m}}. Here, ‘i’ is the simulation batch number and ‘m’ is the total number of observed cellular states or the size of maximum information content possible to be observed in the attractor distribution Π(A) generated in the simulation draw Bᵢ.

If the probability mass functions of all the elements of the sets of cellular states (Cᵢ) drawn from simulation batches were taken into consideration, then according to the “principle of maximum entropy” the probability mass function with highest information entropy will be chosen as the “proper one” for further data analyses. The information entropy of a given batch of simulation can be calculated by calculating the Shannon entropy score of the observed cellular states.

Shannon entropy H(Cᵢ) of the i^{th} instance of a simulation containing the distribution of cellular states Cᵢ={Cᵢ¹, Cᵢ², Cᵢ³, . . . , Cᵢ^{m}} is the negative logarithm of the probability mass function of the observed cellular states in that instance of simulation.

$$\begin{matrix}Hence , Shannon Entropy = H ( {C}_{i} ) = E [ - ln ( P ( {C}_{i} ) ) ] \\ = - {\sum }_{m = 1}^{m} P ( {C}_{i}^{m} ) . {log}_{2} P ( {C}_{i}^{m} )\end{matrix} \qquad ( 3 )$$

Theorem: If Π(A₁), Π(A₂), Π(A₃), . . . , Π(Aₙ) are the attractor distributions generated from initial input states Π(I₁), Π(I₂), Π(I₃), . . . , Π(Iₙ) and their corresponding normalized frequency distributions of cellular states are Π(C₁), Π(C₂), Π(C₃), . . . , Π(Cₙ), then the simulation instance containing highest Shannon entropy score Π(Cᵢ) will possess maximum numbers of different types of cellular states Cᵢ={Cᵢ¹, Cᵢ², Cᵢ³, . . . , Cᵢ^{m}} (Proof not shown here). At the level of maximum Shannon entropy score, there exists a maximum number of distinct cellular states (i.e. max (m)).

Activity Ratio Score: This scoring technique is a metric to determine the contribution of an arbitrary pathway molecule (Xᵢ) in the cell-signaling network to drive cellular dynamics towards a particular cellular state. This scoring technique is useful for extracting important proteins from the set of input proteins, which help the signaling cascade to reach the specific phenotype. In the pathway simulation, given a set of total random input sequences (N), if Sₖ is the total number of random input sequences which direct the model simulation towards a particular cellular state (Cᵢ^{m}), then the activity ratio of any arbitrary input molecule (Xᵢ) is calculated by using the following equations (Eq. 4 and 5).

$$\begin{matrix} Activity Ratio = {( A R )}_{{X}_{i} ∈ X \vert {C}^{j} ∈ C} = {{Log}_{2} ( {e}^{{γ}_{{X}_{i}}} )}_{{X}_{i} ∈ X \vert {C}^{j} ∈ C} & ( 4 ) \\ {( γ )}_{{X}_{i} ∈ X \vert {C}_{j} ∈ C} = {\frac{1}{\vert {S}_{k} \vert} [ {\sum }_{l = 1}^{\vert {s}_{k} \vert} ( {Y}_{l} : {X}_{i} = 1 ) - {\sum }_{l = 1}^{\vert {s}_{k} \vert} ( {Y}_{l} : {X}_{i} = 0 ) ]}_{{X}_{i} ∈ X \vert {C}_{j ∈ C}} & ( 5 )\end{matrix}$$

Let us assume, C:=Set of Cellular States={C¹, C², C³, C^{m}}
m=Total Number Phenotypes observed
Xᵢ∈X:=Set of Input Molecules={X₁, X₂, X₃, . . . , X_{N1}}
N1=Total Input proteins
Y:=Set of Input sequences={Y₁, Y₂, Y₃, . . . , Y_{|S}_{ₖ}_{|}} reaching to cellular state C^{j}
Where, |Sₖ|=Total number of input sequences reaching to a particular cellular states out of N random sequences i.e. |Sₖ|⊆N

Here, the proportion (γ)_{X}_{ᵢ}_{∈X|C}_{ⱼ}_{∈C} of an input protein (Xᵢ) in a given cellular state (Cᵢ) is defined as the ratio of the difference of the number of times the input protein is up-regulated and down-regulated to the total number random input sequences directed towards a specific cellular state in the simulation. In the input list, if a particular protein is essential for a particular cellular state, then its expression was constitutively 1 in the set of all random sequences S_{K}. In that case, the numerator of the proportion (γ)_{X}_{ᵢ}_{∈X|C}_{ⱼ}_{∈C} will be equal to |Sₖ| and maximum and thus the value of the proportion will be equal to +1. Similarly, for a protein, which is inhibitor of a particular cellular state, it can be shown that the proportion will be equal to −1. The Log₂ of the exponential of this proportion value was simply taken as scaling factor to stretch the distributions of the scores in the region of −1.44 to +1.44. This scaling is specifically useful for gaining high resolutions among activity patterns of a large set of input proteins in the development of a particular cellular state. If AR score of a protein is either +1.44 or −1.44 in a given cellular state, then it can be said that the specific protein is highly essential and positive or negative inducer for that particular cellular state.

### Example 5

### Calculation of Phenotype Cost Function and Predictor Scores

The emergence route of intra-tumor heterogeneity and the sub-clones of GBM tumor cells from a common origin of mutated cells were analyzed using tumor cell evolution phylogeny. If the overall expressions of all the pathway molecules at t^{th} time are considered as a ‘state’ of the cell Zₜ={Mₜ¹, Mₜ², Mₜ², . . . Mₜ^{m}:m=Total molecules}, then the entire developmental routes through which different states {Zₜ, Zₜ₊₁, Zₜ₊₂, . . . Z_{t+T}:T=Total simulation time} reach at the attractor states (singleton or periodic) are called as ‘State Transition Graph’(STG). The nodes of a STG are the states and edges that are directed which defines transition of one state to another state at every Boolean update. Hence, STG was able to depict the expression dynamics of all pathway components and the transformation of cells towards different cell types, starting from a common origin of tumor initiating cells. It is observed that for reaching at a particular attractor state, STG has to pass through few transition/intermediate states or nodes {Sₜ, Sₜ₊₁, Sₜ₊₂, . . . } and then land into either fixed-point node {Sₜ₊ₛ} or cyclic attractor nodes{Lₜ₊₁, Lₜ₊₂, Lₜ₊₃, . . . Lₜ₊ₗ}. It is considered that the transition from one state to another state of a cell is also associated with a signaling cost function (i.e. for chemical reactions/any physical processes) and it affects the overall state transition rate. Hence, it was assumed that the probability of a particular attractor/cellular state (Cᵢ) in equilibrium state starting from initial states depends on the overall distance and the total number of molecular transitions to reach the particular state. Hence, to quantify the average signaling cost functions (Φ_{C}_{ᵢ}) in each simulation batch for each cellular state was considered to be the function of total transitions steps and periodicity (in case of cyclic attractor), and the total number of molecular alterations performed starting from initial state to the attractor state. This function is defined in Eq. 6.

$$\begin{matrix}{Φ}_{{C}_{i}} = \frac{ϕ ( {C}_{i} )}{{P}_{{C}_{i}}} & ( 6 ) \\ Where , ϕ ( {C}_{i} ) = \frac{{\sum }_{t = 0}^{s} {d}_{{S}_{t} \to {S}_{r + 1}}}{s} + \frac{{\sum }_{t = 0}^{l} {d}_{{L}_{t} \to {L}_{t + 1}}}{l} & ( 7 )\end{matrix}$$

Where, s=Total number of transition states require to reach at the steady state;

$$l = 1 for Singleton attractor \& {\sum }_{t = 0}^{l} {d}_{{L}_{t} \to {L}_{t + 1}} = 0$$

>1 for Periodic attractor (=Periodicities of the cyclic attractor)
μ=Total mutational costs of the entire model
d_{S}_{ₜ}_{→S}_{ₜ₊₁} & d_{L}_{ₜ}_{→L}_{ₜ₊₁}=Hamming distance between two states in the transient and periodic paths
P_{C}_{ᵢ}=Total number of occurrences of Cᵢ in a simulation batch

Calculation of “Hamming distance” between two successive nodes in STG defines temporal changes or evolution of expression dynamics of pathway molecules in successive time points. On assuming that Sᵢ={X¹ₜ, X²ₜ, X³ₜ . . . , X^{N}ₜ}& Sₜ₊₁={X¹ₜ₊₁, X²ₜ₊₁, X³ₜ₊₁ . . . , X^{N}ₜ₊₁} are the expression vectors referring to arbitrary successive states in STG. The expression of elements (X^{k}ⱼ) lies in binary domain i.e. {0, 1}. The Hamming distance (|d_{S}_{ⱼ}_{→S}_{ⱼ₊₁}|) can be calculated by using the following Eq. 8.

$$\vert {d}_{{S}_{j} \to {S}_{j + 1}} \vert = {\sum }_{k = 1}^{N} ( {X}_{j + 1}^{k} - {X}_{j}^{k} ) \qquad ( 8 )$$

It is also assumed that, apart from the signaling cost a mutated model in which few molecules are constitutively activated/suppressed will also induce mutational cost (Ω) in the cells at the time of its transitions. The mutational cost function for a specific model is dependent on the total number of induced mutations (μ) and the change in total number of added and omitted cellular states in the mutated model with respect to the non-mutated model (μ=0).

Considering the non-mutated model, there are a total maximum X number of cellular states (Cⁿ:C₁ⁿ, C₂ⁿ, C₃ⁿ, . . . C_{X}ⁿ) and in the mutated model there are total maximum Y number of Cellular states (C^{m}:C₁^{m}, C₂^{m}, C₃^{m}, . . . C_{X}^{m}). Suppose in the mutated model there are total μ mutations induced. Hence, the mutational cost (Ω) is defined as follows (Eq. 9).

$$Ω = μ \times \frac{\vert {C}^{n} - ( {C}^{n} ⋂ {C}^{m} ) \vert + \vert {C}^{m} - ( {C}^{n} ⋂ {C}^{m} ) \vert}{( \vert {C}^{n} ⋃ {C}^{m} \vert )} \qquad ( 9 )$$

μ=Total induced mutations in the mutated model
|Cⁿ∪C^{m}|=Total number of cellular states
|Cⁿ−(Cⁿ∩C^{m})|=Total number of cellular states which are not appeared after inducing mutation
|C^{m}−(Cⁿ∩C^{m})|=Total number of cellular states which are newly appeared after inducing mutation

The phenotype cost function (Ψ_{C}_{ᵢ}) is defined as the sum of total signal cost and the mutational cost (if any) for a particular state (Cᵢ) in the STG of a Boolean model. It is defined in Eq. 10.

Ψ_{C}_{ᵢ}=Φ_{C}_{ᵢ}+Ω (10)

Phenotype Predictor Score: The predictor score[Θ(Cᵢ)] of a particular cellular state observed in the simulation is defined as the ratio of the total number of observed occurrences (P_{C}_{ᵢ}) to the total cost (Tc) required for reaching at i^{th} cellular state (Cᵢ) in the attractor space. Hence, this parameter can be defined as follows (Eq. 11).

$$Θ ( {C}_{i} ) = \frac{E ( {P}_{{C}_{i}} )}{E ( {Ψ}_{{C}_{i}} )} \qquad ( 11 )$$

Where, E(P_{C}_{ᵢ}) & E(ΨCᵢ)=Expected values of P_{C}_{ᵢ} and Ψ_{C}_{ᵢ} out of N number of simulation batches

### Example 6

### Maximum Number of Attractor States Using Shannon Entropy in aNSC Model

In the aNSC model simulation, expected normalized frequencies and distributions of cellular states under the steady state situation of each simulation batch were found to be highly uncertain. By considering all distinct cellular states appearing in each simulation batch as “events,” Shannon entropy scores are calculated for every batch. The maximum Shannon entropy score, 1.456 Shannon was subsequently opted for further analyses. Selection of the simulation batch with highest information entropy asserted that the probability of obtaining maximum number of attractor or cellular states with highest normalized frequency is maximum, indicating that 10,000 random initial states used in each batch are sufficient enough to explore maximum number possible attractor states of the model.

### Example 7

### aNSCs are Biased Towards Development of Quiescent, Neural Progenitor and Apoptotic States

Analysis of simulation results (FIG. 6A) revealed that normalized frequencies of singleton attractor or cellular states were comparably higher than periodic attractor states (FIG. 6B), and the singleton attractor state “Apoptosis” has highest normalized frequency (Mean 0.494±0.005 S.D.) compared to other cellular states. The normalized frequency of the undifferentiated “Quiescent state” (Mean 0.378±0.005 S.D.) is the next highest cellular state after apoptosis (FIG. 6B), where a significant large quantity of quiescent neural stem cells (qNSCs) in the neurogenic niche was observed.

Complete differentiation of aNSCs into matured neurons (i.e. NPC Differentiation state) was also observed in this simulation within the singleton attractor state. The normalized frequency (Mean 0.125±0.003 S.D.) of this cellular state observed in the simulation outcomes indicated the natural bias of aNSCs to differentiation of a matured neuron. In contrast, normalized frequency of normal ASPC differentiation state, obtained within periodic attractor space, was observed to be very less (Mean 0.00023±0.00014 S.D.) and was designated as a non-biased event during neurogenesis (FIG. 6B). Hence, it was concluded that the inertia in the internal circuitry of aNSCs drives stem cells to opt neuronal cell fate decision over normal ASPC differentiation process. It was also observed that normalized frequencies of other cyclic cellular states associated with the periodic attractors are also very less as compared to the singleton attractor states (FIG. 6B).

It was also observed that all the periodic cellular states mapped with the self-renewal process of aNSCs followed by apoptosis, NPC differentiation or both (viz. NSC Renewal/Apoptosis, NSC Renewal/NPC Differentiation, NSC Renewal/NPC Differentiation/Apoptosis) have similar normalized frequency values (FIG. 6B). These comparative analyses of cyclic cellular states strongly establish the earlier observations of the general tendency of aNSCs to maintain either of its sternness property or neuronal cell fate specification over the differentiation of astrocytes. Specification of these cell lineages at the time of aNSCs development are found to be associated with the expression profiles of marker proteins (Table 1). In depth, analyses are further performed to distinguish the cell lineages by assessing expression dynamics of these marker proteins. The state transition dynamics of these observed cellular states and the temporal expression patterns of their corresponding marker proteins are disclosed in FIGS. 3 and 4.

### Example 8

### Activity Ratio Scores for Extracting Driver Proteins at Different Cellular States

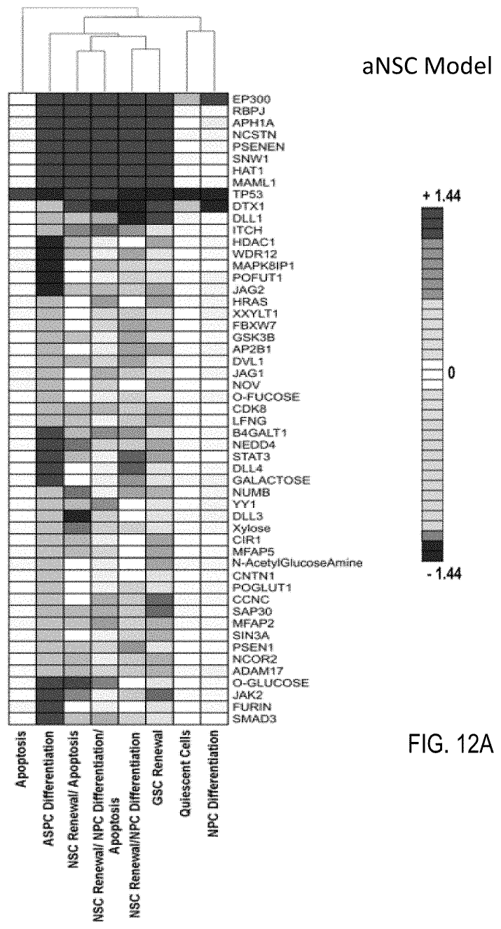

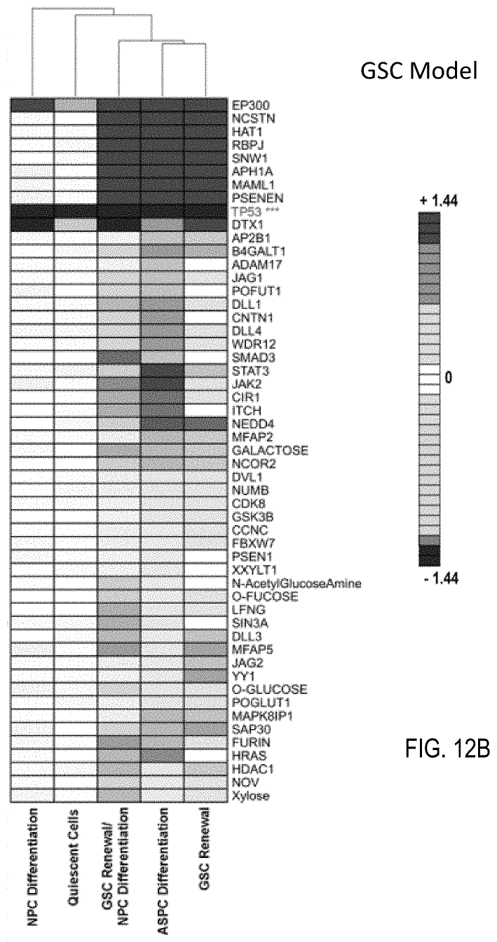

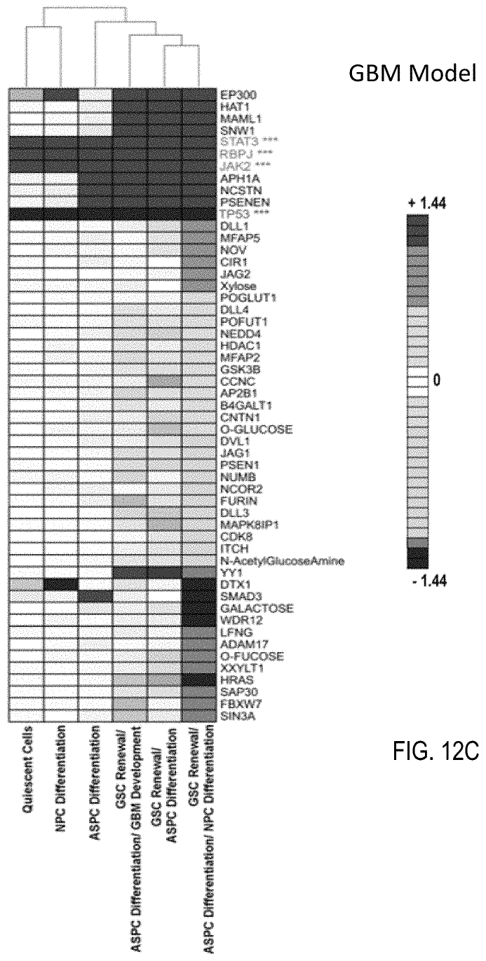

To identify the driver genes/proteins of a particular cellular state, input molecules having optimum “Activity Ratio” score (−1.44≤AR Score≤+1.44) were extracted in each cellular state through comparative analysis (FIG. 12A). It correctly identified important proteins, driving the dynamics of apoptosis, self-renewal and differentiation of adult NSCs. Also, the hierarchical clustering of AR scores of all the cellular states shows separate clusters of fixed-point and cyclic attractor states. It was observed that EP300, RBPJ, APH1A, NCSTN, PSENEN, SNW1, HAT1, and MAML1 having maximum AR scores were highly expressed in all cyclic attractor states. These proteins are core components of Notch pathway, which maintain the balance between self-renewal and differentiations of aNSCs by helping transcriptions of target genes (HES 1-7/HEY 1, 2, L). Higher AR scores of all these proteins were found in self-renewing or cell division cycles (i.e. aNSC renewal state), whereas lower scores of canonical (i.e. core components) and non-canonical (i.e. DTX1) components were observed in neuronal (NPC) differentiation process. On the other hand, higher AR scores of all active Notch pathway components with JAK2/STAT3 proteins were detected in astrocytes differentiation process. The AR score correctly predicted proteins implicated in different sub-types of brain cells and thus justifies its application for identification of driver proteins behind development of a particular phenotype or cellular state.

### Example 9

### Phenotype Predictor Scores to Predict the Appearances of Cellular States

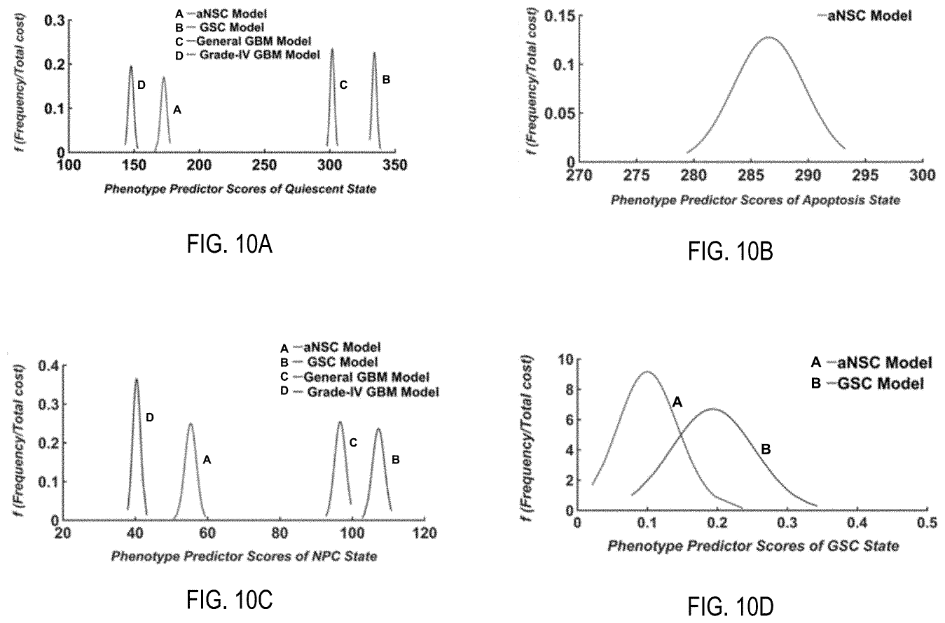

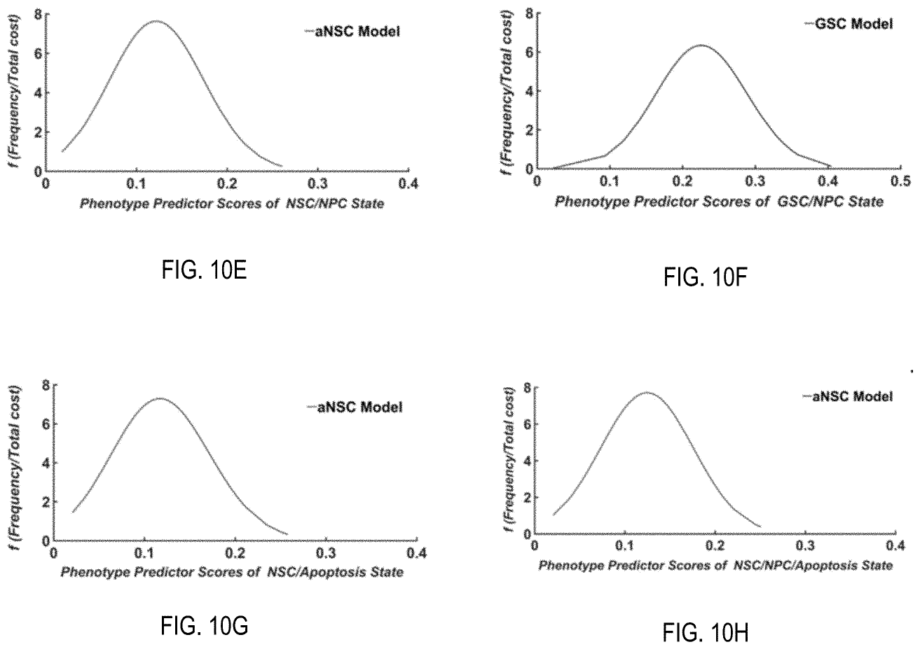

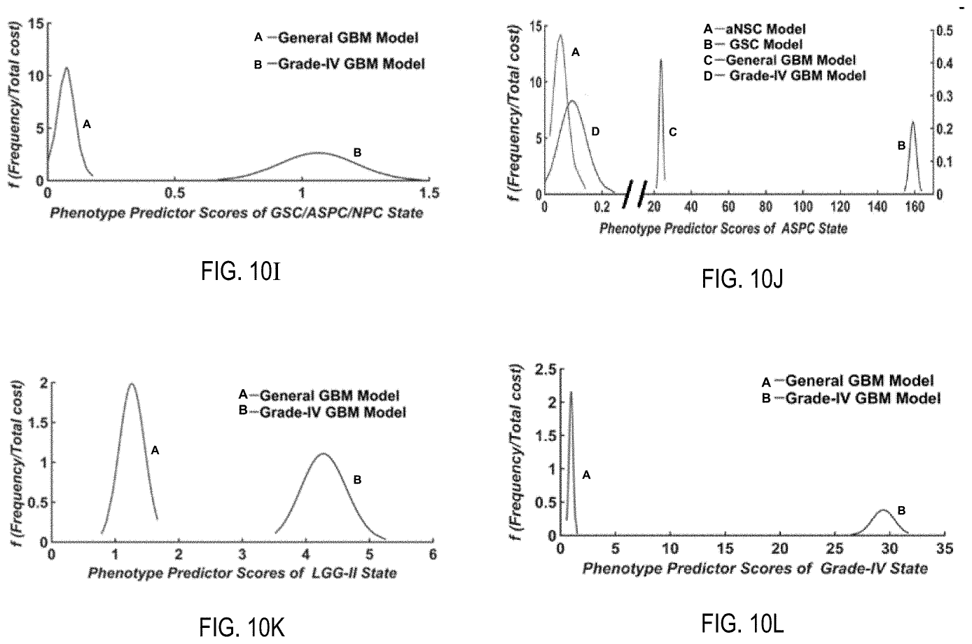

A higher value of phenotype predictor score of an arbitrary cellular state signifies the greater chance of that cellular state to appear within the attractor space. The mean values with 95% CI of all the cellular states observed in aNSC, GSC, general GBM and Grade-IV models are provided in Table 2, which follow Cauchy-like distribution (FIG. 10).

TABLE 2
Mean values with 95% Confidence Interval of the
phenotype predictor scores of cellular states

| | Models | | | |
|---|---|---|---|---|
| | aNSC | GSC | General GBM | Grade-IV GBM |
| Observed Cellular | Mean | Mean | Mean | Mean |
| States or Phenotypes | [95% CI] | [95% CI] | [95% CI] | [95% CI] |
| Quiescent | 172.813 | 334.160 | 301.827 | 147.688 |
| | [171.596 | [333.246 | [300.945 | [146.630 |
| | 174.032] | 335.074] | 302.709] | 148.747] |
| Apoptosis | 286.581 | NA | NA | NA |
| | [284.957 | | | |
| | 288.206] | | | |
| NPC | 55.326 | 107.264 | 96.677 | 40.372 |
| Differentiation | [54.496 | [106.388 | [95.862 | [39.804 |
| | 56.156] | 108.140] | 97.492] | 40.940] |
| GSC Renewal | 0.100 | 0.193 | NA | NA |
| | [0.078 | [0.163 | | |
| | 0.124] | 0.224] | | |
| NSC Renewal/ | 0.123 | NA | NA | NA |
| NPC Differentiation | [0.095 | | | |
| | 0.150] | | | |
| GSC Renewal/ | NA | 0.226 | NA | NA |
| NPC Differentiation | | [0.194 | | |
| | | 0.259] | | |
| NSC Renewal/ | 0.118 | NA | NA | NA |
| Apoptosis | [0.090 | | | |
| | 0.147] | | | |
| NSC Renewal/ | 0.124 | NA | NA | NA |
| NPC Differentiation/ | [0.098 | | | |
| Apoptosis | 0.151] | | | |
| GSC Renewal/ | NA | NA | 0.072 | 1.063 |
| ASPC Differentiation/ | | | [0.053 | [0.984 |
| NPC Differentiation | | | 0.0917] | 1.141] |
| ASPC Differentiation | 0.054 | 0.098 | 23.655 | 159.059 |
| (LGG-I) | [0.040 | [0.073 | [23.150 | [158.118 |
| | 0.069] | 0.123] | 24.160] | 160.000] |
| GSC Renewal/ | NA | NA | 1.265 | 4.282 |
| ASPC Differentiation | | | [1.161 | [4.096 |
| (LGG-II) | | | 1.370] | 4.470] |
| GSC Renewal/ | NA | NA | 0.970 | 29.414 |
| ASPC Differentiation/ | | | [0.873 | [28.875 |
| GBM Development | | | 1.066] | 29.955] |
| (Grade-IV) | | | | |

It was observed that the mean phenotype predictor score (mean 147.688, 95% CI [146.630 148.747]) of the “Quiescent” cellular state is lowest in the highly mutated Grade-IV GBM as compared to the other models. These results were also in accordance with FIG. 12A, in which lower normalized frequency of quiescent state in Grade-IV GBM model is detected as compared to general GBM model. Therefore, it can be stated that the percentage of quiescent cells will be less in the heavily mutated tumorigenic niche (i.e. Grade-IV GBM cells) as compared to normal neurogenic niche (adult NSCs). A similar result is also observed for “NPC differentiation” state, which shows that in the same tumorigenic niche, the heavily mutated tumor cells are least influenced (mean 40.372, 95% CI [39.804 40.940] to differentiate into matured neurons as compared to normal aNSCs in the neurogenic niche. The score for GSC renewal (mean 0.193, 95% CI [0.163 0.224]) state is also higher in the GSC model as compared to the aNSC model (mean 0.100, 95% CI [0.078 0.124]). A comparative analysis of LGG-I, LGG-II, and Grade-IV cellular states observed in general GBM and Grade-IV GBM models reveal that the phenotype predictor scores for all of these cellular states are comparably higher in Grade-IV GBM model as compared to other model simulations. For example, the score for Grade-IV cellular state in general GBM model is less (mean 0.970, 95% CI [0.873 1.066]) as compared to Grade-IV GBM model (mean 29.414, 95% CI [28.875 29.955]). Hence, it is proven that the induced alterations of the protein expressions in Grade-IV GBM models can accelerate the growth of LGG-I, LGG-II and high grade (Grade-IV) glioblastoma cells, which in turn help to predict the chances of occurrences of the oncogenic cells in the tumor ecosystem.

### Example 10

### Case Study Using TCGA-LGG & TCGA-GBM RNASeq Samples Data

### (i) Selection of Patient Cohorts and Preparation of RNASeq Sample Data Sets

Two patient cohorts consisting of low-grade (TCGA-LGG) and high-grade Glioblastoma (TCGA-GBM) from “The Cancer Genome Atlas (TCGA)” research networks were selected. HTSeq raw counts files (RNASeq experiment) of Glioblastoma patients with TP53 mutation were selected to determine mRNA expression profiles of these two patient cohorts. The following is the statistics of the two cohorts from which the RNASeq raw count data was downloaded on 27th Apr., 2017 (Table 3).

TABLE 3
Statistics of the TCGA Glioblastoma patient cohorts

| | | | | | # of Samples (RNA-Seq Raw Counts | | | |
|---|---|---|---|---|---|---|---|---|
| | | | | | # | # | # | |
| | | | | | Normal | Primary | Recurrent | |
| | # of Cases (Patients) | | | | Tumor | Tumor | Tumor | |
| Cohorts | M | F | U | Total | Samples | Samples | Samples | Total |
| TCGA-LGG | 282 | 228 | 1 | 511 | 0 | 513 | 16 | 529 |
| (General) | | | | | | | | |
| TCGA-LGG | 141 | 100 | 1 | 242 | 0 | 240 | 15 | 255 |
| (TP53 Mutated) | | | | | | | | |
| TCGA-GBM | 366 | 230 | 21 | 617 | 5 | 13 | 156 | 174 |
| (General) | | | | | | | | |
| TCGA-GBM | 32 | 21 | 0 | 53 | 0 | 57 | 0 | 57 |
| (TP53 Mutated) | | | | | | | | |

M = Male,
F = Female,
U = Undefined/Not mentioned

The TCGA-Low Grade Glioblastoma (TCGA-LGG) cohort contains tumor samples from Grade-II and Grade-III glioblastoma patients, whereas the TCGA-GBM cohort contains samples from Grade-IV tumor patients. The RNASeq raw counts data (available as TXT files) for each patient is available in the open source GDC Data portal of National Cancer Institute (https://portal.gdc.cancer.gov/). Further information about the workflows related to sample collection; mRNA sequencing and data processing, read alignments, and mRNA quantification etc. are available in GDC Data User's Guide (https://docs.gdc.cancer.gov/Data/PDF/Data_UG.pdf).

(ii) Differential Expression Analyses of mRNA Molecules

The RNA SEQ raw counts data files corresponding to the patient cohort, which have TP53 mutation, are extracted from TCGA-LGG (General) and TCGA-GBM (General) patient cohorts. Differential expression analyses are performed by forming the contrasts between primary (TP53 mutated) (i) LGG (total samples=240) and (ii) GBM (total samples=57) tumor samples versus solid normal tumor (total samples=5) samples using “edgeR” statistical package. The mRNA molecules of following proteins from the set of input proteins (not provided herein) are found to be significantly expressed (up or down) in differential expression analyses (Table 4). The mRNA expression patterns observed in this analysis were considered as transcriptomics profiles of two different patient cohorts.

TABLE 4
Differentiation expression of the mRNA molecules in two different patient cohorts

| TCGA-GBM (TP53 Mutation) vs. | | TCGA-LGG (TP53 Mutation) vs. | |
|---|---|---|---|
| Normal solid tumor | | Normal solid tumor | |
| Up Regulation | Down Regulation | Up Regulation | Down Regulation |
| APH1, DLL3, FRINGE, | CNTN1, DVL, DTX1, | DLL1, DLL3, | FBW7, JAG2, NOV |
| GASE, HAT, HDAC, | FBW7, JAG2, JIP1 | FRINGE, | |
| JAG1, MAGP1, | | NEDD4, | |
| MAGP2, NEDD4, | | POFUT1 | |
| POFUT1, SAP30 | | | |

(iii) Logical Simulations Using TCGA-LGG and TCGA-GBM Transcriptomics Data

The transcriptomics profiles of the input protein molecules observed in TCGA-LGG and TCGA-GBM patient cohorts were taken as inputs for further simulations of aNSC and general GBM model simulations.

### (iv) Analysis of TCGA-LGG Patient Cohort

In the TCGA-LGG patient cohort there were total 5 and 3 protein molecules found to be over and under expressed, respectively (Table 2). Mutation or down regulation of P53 protein is also considered. Hence, out of total 53 input molecules of the master aNSC model, there were a total of 9 protein molecules kept constitutively expressed at ON or OFF state and eliminated from the input list for further randomization in the new simulation. Hence, the total mutations introduced in TCGA-LGG transcriptomics data on master aNSC model is 9 and rest 44 input proteins were randomized 10000 times in 10 separate batches.

The mean normalized frequency values of each cellular state were calculated from these 10 independent simulation batches, which were further used for checking the goodness-of-fit with normalized frequency values observed for cellular states in the master aNSC model. Chi-square goodness of fit test is used to compare the normalized frequency distributions of observed normalized frequencies with expected normalized frequencies observed in the master aNSC model. This statistical test shows significant differences between the expected and observed data (P-value <0.001), which in turn proves that the transcriptomics profile extracted from the TCGA-LGG cohort do not indicate the development of normal neurogenesis of aNSCs (FIG. 12A).

Due to the imposed mutations such as P53 down-regulation, DLL1, DLL3 up-regulation in the new model, cellular states “Apoptosis” and “NSC/NPC/Apoptosis” were not found in the attractor space. On the other hand, the normalized frequencies of the cellular states “ASPC differentiation” and “GSC renewal” were found in higher numbers in the TCGA-LGG transcriptomics data model of aNSC. Hence, it proves that the neural stem cells having transcriptomics profile of TCGA-LGG cohort were more inclined to the development of glioblastoma stem-like (GSC) and mutated astrocytes or tumor cells (ASPC).

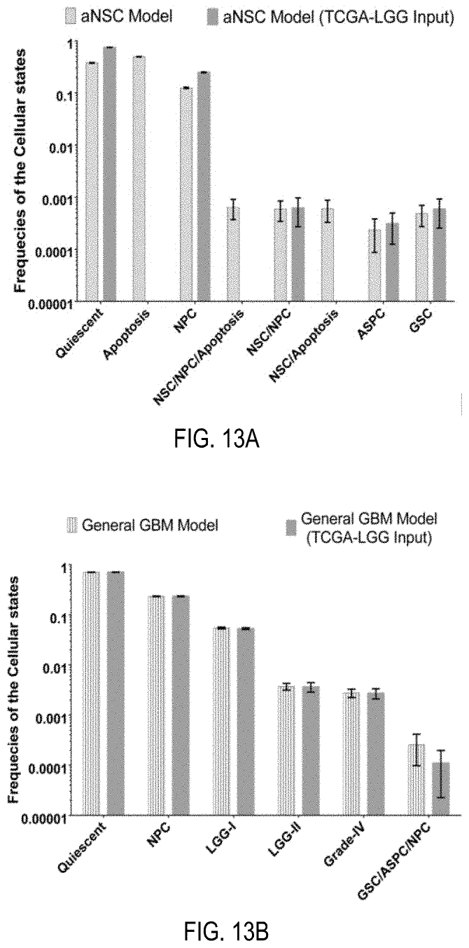

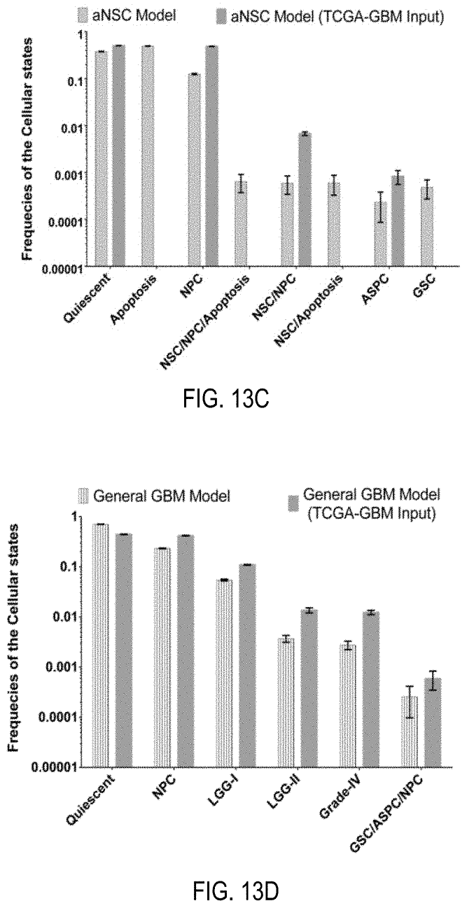

The comparative statistics of the normalized frequency distributions of each cellular state observed in these two simulations is represented in FIG. 13A. Following this simulation, the TCGA-LGG transcriptomics data is considered as inputs in the previously developed, master model of general GBM development. In the master GBM model, there are already 4 mutations added (including P53) and 8 mutations are further added as per the TCGA-LGG transcriptomics expression profile (Table 2). Hence, altogether there are total 12 mutations (μ) introduced in the master general GBM model and a new derivative model is developed by keeping these 12 input proteins at constitutively up or down regulated states. These 12 input proteins are kept aside for further randomization and the rest 41 input proteins are randomized 10000 times in 10 separate batches.

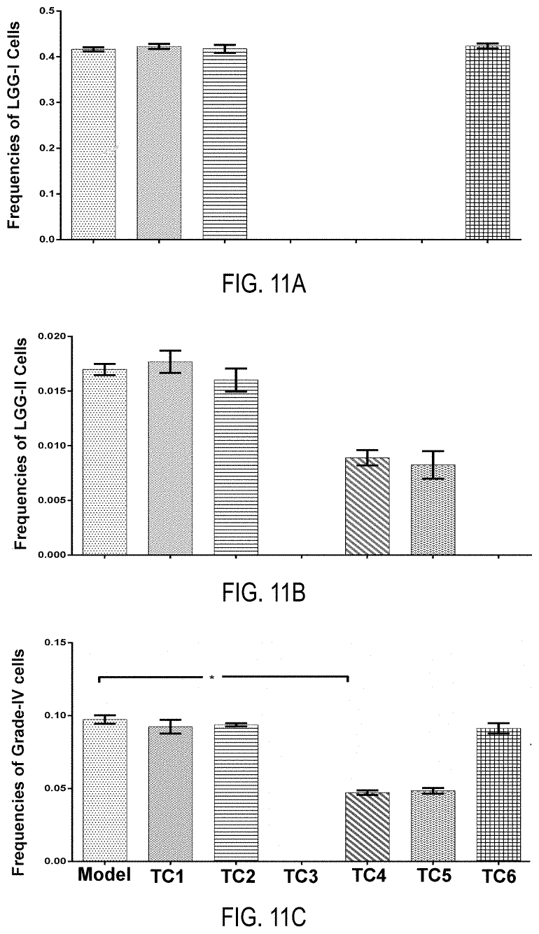

The mean values of the phenotype predictor scores of the tumorigenic cellular states viz. LGG-I (mean 19.00, 95% CI [18.60 19.41]), LGG-II (mean 1.10, 95% CI [0.98 1.22]), and Grade-IV (mean 0.83, 95% CI [0.73 0.93]) observed in this new simulation are similar with the values observed in master general GBM model (Table 2). The comparative statistics of the normalized frequency distributions of each cellular state observed in these two simulations is represented in FIG. 11B.

### (v) Analyses of TCGA-GBM Patient Cohort

Similar to the analyses of TCGA-LGG tumor samples cohort, the transcriptomics profile (Table 2) observed for the TCGA-GBM sample cohort is also considered at first as inputs in the master aNSC and general GBM models. Total 12 and 6 proteins are found to be up and down regulated, respectively, in the differential expression analyses in the tumor sample cohort with identified TP53 mutation. Therefore, in total 19 mutations (μ=12+6+1) of the input proteins are considered in the input in the master aNSC model and a new model using the TCGA-GBM transcriptomics data on master aNSC model is developed. Here, the extra 1 mutation is added for the P53 mutation. These 19 input proteins are kept constitutively at ON or OFF state as per the differentiation expression results (Table 2) and the rest 34 input proteins out of 53 are further randomized 10000 times in 10 separate simulation batches. Similar Chi-square goodness-of-fit test is performed here to assess the similarities of the normalized frequency values of each cellular state observed in master aNSC and new model simulations. Simulation results and the comparative statistics of the normalized frequency distributions between these two models are depicted in FIG. 11C.

On the other hand, another set of simulation is performed to examine the effects of the differentially expressed transcripts of the TCGA-GBM tumor samples cohort (Table 2) on the master general GBM model. Here, the master general GBM model contains 4 mutations (TP53, JAK2, STAT3, and RBPJ) and in the TCGA-GBM cohort another 18 proteins are found to be differentially expressed. Therefore, in total there are 22 mutations (=18+4) will be added in the new simulation. These 22 proteins are kept constitutively expressed (either up or down) based on the transcriptomics profile generated from differential expression analyses (Table 2) and the rest 31 out of 53 input proteins are randomized 10000 times in 10 separate batches. Similar Chi-square goodness-of-fit test is also performed to study the similarities of the normalized frequency values of each cellular state observed in master general GBM model and new model simulations. A comparative statistic of the cellular states observed in this new model as compared to the master general GBM model is represented in FIG. 11D.

### Example 11

### Methodology for Drug Target Screening

The simulation outcome of “Grade-IV GBM model” simulation shows significant increase in the number of Grade-IV tumor cells in the attractor space. The cellular state involved is GSC Renewal/ASPC Differentiation/GBM Development (FIG. 12C). The periodic state transition dynamic in the steady state level observed in the cellular state mainly occurs due to cyclic expression patterns of its corresponding marker proteins. A subset of intracellular intermediate molecules strongly tuned and correlated with the activity pattern of Grade-IV cellular state via marker proteins were the most significant molecules for sustainment of this cellular state. It is expected that perturbing expressions (i.e. logical states) of such molecules (individually or in combination) will alter the activity pattern of the Grade-IV cellular state and those proteins will be considered as potential drug targets. However, identification of such small subset of molecules out of the large set of modelled pathway molecules is a challenging task.

The intermediate molecules which are mutually interconnected and highly correlated with the temporal activity pattern observed for Grade-IV cellular state in the “Grade-IV GBM” model simulation study are required to be extracted. The correlation and delay between a pair of time course data could be calculated by using Fast Fourier Transform (FFT) analysis. Hence, the delay and pair-wise correlation between the activity patterns of Grade-IV tumorigenic cellular state (i.e. target signal) and the time-course logical expressions data of all the intermediated molecules (i.e. Query signal) are measured by the following method.

Considering that C^{Grade-IV}={c₁, c₂, c₃, . . . , c_{T}} is the time series activity (ON or 1 and OFF or 0) profile of the Grade-IV cellular state observed in the STG of “Grade-IV GBM model” simulation at the discrete time points t=1, 2, 3, . . . , T. This time-course data of Grade-IV cellular state is considered as the “Target” signal. Similarly, let us consider that the time-course logical expression (ON/1, OFF/0) profile of any arbitrary molecule Xᵢ={x₁, x₂, x₃, . . . , X_{T}}, which is considered as “Query” signal. Both the temporal signals (S) are decomposed into cyclic patterns (i.e. frequency domain) with each frequency n=1, 2, 3, . . . , T−1 by following FFT analyses as shown in Eq. 13 & Eq. 14.

$$\begin{matrix}{C}_{n} = \frac{1}{T} {\sum }_{n = 1}^{T - 1} {c}_{t} {e}^{- i 2 π n \frac{t}{T}} & ( 13 ) \\ {X}_{n} = \frac{1}{T} {\sum }_{n = 1}^{T - 1} {x}_{t} {e}^{- i 2 π n \frac{t}{T}} & ( 14 )\end{matrix}$$

Amplitude of the cycle with frequency n=0 is neglected. The amplitudes and phase angles of the cycles with higher frequencies (n>0) are calculated for both the signals. The frequency of the cycle (n) for which the amplitude is found at maximum magnitude is at first identified. After that phase angles of the cycles from both the target and query signals (ϕ_{C}ⁿ,ϕ_{X}ⁿ) are calculated at that frequency (n) and the difference or delay Δⁿ_{CX}=ϕ_{C}ⁿ−ϕ_{X}ⁿ between the two signals is measured. The delay between two signals δ_{CX} is further calculated in the range of

$$0 to \frac{T}{n}$$

by using the following Eq. 15.

$${δ}_{CX} = \frac{{Δ}_{CX}^{n}}{( \frac{360 n}{T} )} \qquad ( 15 )$$

Lagged Pearson correlation coefficient is also calculated for measuring the strength and association between the two signals or trajectories. In this work the delay and correlation between Grade-IV cellular state and for each pathway molecules are measured pair-wise. There are total six probable outcomes, which are found while comparing all such pairs of trajectories (i.e. Target vs. Query) using this approach. These entire mathematical calculations are done in “DynOmics” package developed in the statistical package “R”.

(i) The signals are positively correlated (correlation >0) with no delay (Delay=0). In this case both target and query signals are superimposed on each other and the phase (or direction) of the signals is same (i.e. positively correlated). Hence, it is considered that the molecular expression pattern (i.e. query signal) is positively influencing the activity pattern or dynamics of Grade-IV tumor state and there is no delay between the time-course profiles. Therefore, the molecule is a strong positive inducer or activator of Grade-IV tumor cells, which means when the molecule is at ON (i.e. up-regulated/active) state, the activity of Grade-IV cellular state is also High (or ON).

(ii) The signals are positively correlated (correlation >0) with negative delay (Delay <0). In this case the phase (or direction) of both the signals is same (i.e. positively correlated), but the initiation of the target signal at initial time point is delayed with respect to the query signal. Hence, it is considered that the molecular expression pattern (i.e. query signal) is positively influencing the activity pattern or dynamics of Grade-IV tumor state, but there is a lag of the Grade-IV time-course activity profile with the respect to that positively influencing molecule. Therefore, the molecule is a positive inducer or activator of Grade-IV tumor cells, which means activation of these molecules, will lead to the higher expression of Grade-IV tumor state.

(iii) The signals are negatively correlated (correlation <0) with positive delay (Delay >0). In this case the phases of the signals are opposite (i.e. negatively correlated) and the initiation of the target signal is ahead of the query signal at initial time point. Hence, it is considered that the molecular expression pattern (i.e. query signal) is negatively influencing the activity pattern or dynamics of Grade-IV tumor state, but the Grade-IV time-course activity profile is running ahead with the respect to that negatively influencing molecule. Therefore, the molecule is a negative inducer or activator of Grade-IV tumor cells, which means which means when the molecule is at ON (i.e. up-regulated/active) state, the activity of Grade-IV cellular state is Low (or OFF).

(iv) The signals are negatively correlated (correlation <0) with no delay (Delay=0). In this case the phases of the signals are opposite (i.e. negatively correlated), but there is no delay at the initiation of the signals at initial time point. Hence, it is considered that the molecular expression pattern (i.e. query signal) is negatively influencing the activity pattern or dynamics of Grade-IV tumor state, but there is no lag between the trajectories of these two signals. Therefore, the molecule is a strong negative inducer or inhibitor of Grade-IV tumor cells which means activations of these molecules, will inhibit the expression or activation of Grade-IV tumorigenic state.

(v) The signals are negatively correlated (correlation <0) with negatively delay (Delay <0). In this case the phases of the signals are opposite (i.e. negatively correlated), but the initiation of the target signal at initial time point is delayed with respect to the query signal. Hence, it is considered that the molecular expression pattern (i.e. query signal) is negatively influencing the activity pattern or dynamics of Grade-IV tumor state, but there is a lag of the Grade-IV time-course activity profile with the respect to that negatively influencing molecule. Therefore, the molecule is a negative inducer or inhibitor of Grade-IV tumor cells which means activations of these molecules, will inhibit the expression or activation of Grade-IV tumorigenic state.

(vi) The signals are positively correlated (correlation >0) with positive delay (Delay >0).

In this case the phase (or direction) of both the signals is same (i.e. positively correlated), and the initiation of the target signal is ahead of the query signal at initial time point. Hence, it is considered that the molecular expression pattern (i.e. query signal) is positively influencing the activity pattern or dynamics of Grade-IV tumor state, but the Grade-IV time-course activity profile is running ahead with respect to negatively influencing molecule. Therefore, the molecule is a positive inducer or activator of Grade-IV tumor cells which means activations of these molecules, will lead to higher expression of Grade-IV tumor state. The pathway molecules, which show significant positive correlation (correlation value ≥0.6) and Delay ≤3 with the activity trajectory of Grade-IV cellular state, are listed in Table 5.

TABLE 5
Delay difference and significant correlation observed
between Grade-IV trajectory and pathway molecules

| Original | Query | | | |
|---|---|---|---|---|
| Signal | Signal | Delay ≤3 | Correlation >=0.6 | P-Value |
| Grade-IV | GFAP | 1 | 1 | 0 |
| Grade-IV | HIF1A* | 1 | 1 | 0 |
| Grade-IV | NUC_STAT3* | 2 | 1 | 0 |
| Grade-IV | MASH1* | 3 | −1 | 0 |
| Grade-IV | NESTIN | 3 | −1 | 0 |
| Grade-IV | NEUROD | 3 | −1 | 0 |
| Grade-IV | PTEN* | 3 | −1 | 0 |
| Grade-IV | STAT3_P* | 3 | 1 | 0 |
| Grade-IV | BAD | 0 | −0.7698004 | 0.005588 |
| Grade-IV | AKT* | 1 | 0.7637626 | 0.010131 |
| Grade-IV | NGN1* | 2 | −0.7559289 | 0.018452 |
| Grade-IV | PI3K* | 2 | 0.7559289 | 0.018452 |
| Grade-IV | HES1 | 0 | 0.6236096 | 0.040347 |
| Grade-IV | HES5 | 0 | 0.6236096 | 0.040347 |

*Proteins selected for drug target screening as they do not belong to the class of marker proteins responsible for defining different cellular states.

Perturbation study: The molecules listed in Table 5 showing positive correlation with the activity profile of Grade-IV cellular state are suppressed (i.e. targeted) by freezing the expression states as down-regulated (or OFF) state in the “Grade-IV GBM” model. On the other hand, the molecules, which are negatively correlated, are kept up-regulated (i.e. ON) in the perturbation study. The outcomes of targeting these identified proteins (individually or combinations) in terms of normalized frequency distributions are shown in FIG. 11. In Table 5, the molecules which have absolute correlation value ≥0.75 with P value <0.05, Delay ≤3 and shown profound effect of reducing the activity of LGG-I, LGG-II, and Grade-IV cellular states while perturbing their expressions in the “Grade-IV GBM” model are shown rank wise. Here, the activity profiles of LGG-I and LGG-II cellular states are also assessed to analyze the suppressing effects of targeting the selected protein on all sub-types of GBM tumor. There are four groups of drug targets, which are mentioned in Table 4 viz. (i) PI3K/AKT, (ii) STAT3/Nuc_STAT3, (iii) MASH1, and (iv) HIF1A, are considered for further drug target screening and ranking simulation analyses. The simulation results show that the perturbations of STAT3/Nuc_STAT3 or MASH1 proteins individually are highly effective than perturbing the other molecules from the remaining two groups viz. (i) PI3K/AKT and (iv) HIF1A. It can be also concluded that the protein molecules showing maximum delay ≤3 and correlation ≥0.6 with the target signal (i.e. expression dynamics of Grade-IV) can be allowed to screen the suitable proteins as drug targets (Table 5).

In another aspect, transcriptomics data are provided as inputs, a novel predictive model is developed to predict the future risk of Glioblastoma tumor of an individual with appropriate grade by using the transcriptomics profile of that individual as input. Also, a novel technique is introduced to screen and rank the potential drug-targets for suppressing the growth and differentiation of tumor cells.

Thus, methods according to embodiments of this disclosure may be deployed for personalized, glioblastoma related pathological studies, such as risk prediction, tumor grade detection, biomarker identification, and ranking of most potential drug targets by using transcriptomics/proteomics data of glioma or suspected patients, thus identifying identify the combinations of most effective and drug-targetable signaling proteins for the personalized and target-based anti-glioma therapy. The present methods helps to screen and identify potential molecules for target-based anti-glioma therapeutics. Identifying driver proteins in Notch signaling by activity ratio helps to establish the role of Notch pathway as the guardian behind the maintenance of stem-like properties of adult NSCs and the suppressor of neuronal differentiation process thorough this in-silico approach.

Using the methods disclosed herein, the fundamental understandings of the underlying molecular processes of Notch and its cross-talks pathways are explored to assess the governing principles working behind the self-renewal, differentiation, apoptosis, and cell growth arrest (i.e. quiescent state) of aNSCs/GSCs or glioma tumor cells. By assessing the variances in the genetic makeup of individual tumor cells, methods according to embodiments herein are competent to dissect underlying mechanisms working on the emergence of different sub-types and sub-clonal heterogeneities of glioma tumor.

The chances of developing different sub-types of glioma tumor cells from GSCs or astrocytes cells are also quantified, which is further applied as a personalized approach to detect the risk of early onsets of glioma tumor with appropriate grades (or sub-types).

We claim:

1. A method for determining the risk of tumor development grade of Glioblastoma (GBM or Low Grade Glioblastoma) from the general GBM developmental model, the method comprising;
(i) collecting tumor samples from patients diagnosed with glioblastoma cancer followed by extracting and purifying whole cell mRNA form the samples;
(ii) performing high-throughput mRNA sequencing process requiring purified and enriched mRNA library for generating the pair-end short reads of the sequences, which is further checked by measuring the library size and base quality and shared in a computer readable text file;
(iii) processing RNA-Seq files of step (ii) for quality checking of the sequences, aligning of the sequence reads to the human reference genome and quantifying the transcripts/genes;
(iv) performing differential gene expression analyses to determine the differential mRNA expression profile of the collected tumor specimen with respect to normal cells; and
(v) producing a predictive computing module based on the tumor specimen, wherein the predictive computing module utilizes the mRNA expression profile of step (iv) comprising select input proteins, performs mechanistic simulations to compute the frequencies of tumorigenic [LGG (Low Grade Glioblastoma) and GBM] and non-tumorigenic cells, followed by computing a novel quantitative (i.e., phenotype predictor) score of each sub-types of glioma tumor cells to predict the chances of occurrence of glioma tumor of respective grades.

2. The method of claim 1, wherein the method is based on an activity ratio (AR).

3. The method of claim 1, wherein the predictive computing module comprising selecting marker proteins involved in cellular states of phenotypic functions of normal neurogenesis and glioblastoma tumorigenesis in adults is selected from the group consisting of apoptosis, aNSC (Neural Stem Cell) renewal, NPC (Neuron Progenitor Cell) differentiation, ASPC (Astrocyte Progenitor Cell) differentiation, and GBM (Glioblastoma) development, the method comprising:
(i) defining cellular states, viz. quiescent (or inactive) state, NSC (neural stem cell) renewal, GSC (Glioma Stem Cell) renewal, ASPC (Astrocytes progenitor cell), low and high grade glioblastoma tumor (LGG-I, LGG-II, Grade-IV) states based on activation of phenotypic function and co-expression of multiple marker proteins;
(ii) simultaneously identifying
(a) proteins in the network model that drive the cellular states by measuring the novel activity ratio (AR) scores thereby identifying driver proteins causing tumorigenesis in the subject and classifying the tumors into different grades (low and high); and
(b) a risk of a subject developing glioblastoma (low or high) by measuring the novel phenotype predictor score of the cellular states;
(iii) identifying intra-tumor heterogeneities by assessing the variations of protein expressions with high or low grades of glioblastoma tumor cells and the molecular sub-clones of the tumor cells; and
(iv) screening and identifying a combination of cell signaling proteins as potential drug targets by a novel perturbation analysis and determining the chance of tumor relapse or cell death.

4. The method of claim 3, wherein the predictive computing module comprises:
(i) defining a plurality of input, intermediate, and output molecules (e.g., proteins, metabolites) and their intra-cellular biochemical reactions involved in the process of normal neurogenesis and glioblastoma tumorigenesis in adult human brain;
(ii) selecting marker proteins (or end products of the intra-cellular biochemical reactions) responsible for common phenotypic outcomes observed during normal neurogenesis;
(iii) writing logical equations for defining the functional and dynamic relationships between the molecules and their connections with the phenotypic expressions;
(iv) defining a cellular state “Quiescent (inactive)” wherein no phenotypes are observed;
(v) randomly assigning the binary initial states (either 1 or 0) to the input proteins and creating 1 million random expression vectors of the input proteins;
(vi) dividing the 1 million binary expression vectors of the input proteins in 100 separated batches, in which each batch contains 10,000 random expression vectors;
(vii) starting the simulations for each batch (10,000 initial states) and recording the phenotypic output of each initial state;
(viii) iterating the process over all 100 batches and simultaneously recording the distributions of the phenotypic outcomes (or cellular states) of each batch;
(ix) computing the Shannon entropy of the observed phenotypic or cellular outcomes of each simulation batch and selecting the batch with highest entropy value for further analyses to compute the activity ratio (AR) score and frequency distributions of the observed cellular states;
(x) checking the cellular state or phenotypes viz. quiescent state, apoptosis, NSC renewal, NPC differentiation, ASPC differentiation, and GSC renewal have arrived in the distribution or not;
(xi) discarding the results if any of the phenotypes of (x) are missing in the simulation batch and repeating the steps from (iii) to (x) until observing the appropriate distributions of the desired phenotypes or cellular states mentioned in (x) of adult neurogenesis process;
(xii) selecting the simulation batch with highest entropy if the simulation successfully reproduce the normal and aNSC's developmental process by expressing the phenotypes mentioned in (x) and subsequently compute the activity ratio (−1.44≤AR≤+1.44) of each input protein for individual phenotype or cellular state;
(xiii) validating the activity ratio (AR) score of each protein (i.e., positive AR score for up-regulated/active states=+1.44; negative AR score for down-regulated/inactive states=−1.44) representing each phenotype with the experimental data, such as immunohistochemistry, western blot, mass spectrometry, or transcriptomics; and
(xiv) if a protein shows wrong activity in any of the phenotype, repeating the simulation from step (iii) to (xiii), or otherwise proceeding to the next steps:
(a) declaring the proteins with positive (and maximum=+1.44) and negative (and minimum=−1.44) activity ratio (AR) score observed in a particular phenotype as the driver proteins for the development of that phenotype, for example, the positive and maximum AR score of P53 is the driver of the phenotype apoptosis;
(b) naming the simulation strategy from (i) to (xiv) as the model of aNSC development simulation, in which the normal neurogenesis and gliogenesis including natural cell death (apoptosis) processes are successfully simulated and validated with the experimental observations;
(c) fitting the distribution of the phenotype prediction scores of each cellular state observed in 100 simulation batches with different distribution functions and calculating the confidence interval of the phenotype predictor score of a cellular state from its corresponding distribution function;
(d) finding the cellular state Glioblastoma stem cells (GSC) renewal with input protein P53 at inactive state (i.e., mutation or negative AR score) in the simulation outputs and thus proving the inactivation of tumor suppressor protein P53 as the driver of GSC development from aNSCs in human brain; and
(e) removing P53 protein from the input protein list made in step (v) and considering the P53 protein as constitutively down-regulated (binary: 0 or False) state in the logical equation model developed in step (iii) to create another new model.

5. The method of claim 1, wherein the cellular states are selected from the group consisting of quiescent (or inactive) state, NSC (neural stem cell) renewal, GSC (Glioma Stem Cell) renewal, ASPC (Astrocytes progenitor cell), and low and high grade glioblastoma tumor (LGG-I, LGG-II, Grade-IV) states based on activation of phenotypic function and co-expression of multiple marker proteins.

6. The method of claim 4 in a model with P53 mutation comprising:
(i) assessing the observed cellular states and their frequency distributions across 100 simulation batches and identifying the simulation batch with highest Shannon entropy as mentioned in (ix);
(ii) selecting the simulation batch with highest Shannon entropy for activity ration calculation and assessing the distribution of the frequencies of cellular states;
(iii) checking whether the cellular state “apoptosis” is present in the frequency distribution or not. If yes, then modify the logical equations as mentioned at step (iii) and repeat the steps from (iii) to (xvi), otherwise proceed to the next step;
(iv) checking whether the cellular state “GSC renewal” and “ASPC differentiation” are present or not. If not, then modify the logical equations as mentioned at step (iii) and repeat the steps from (iii) to (xvii), otherwise proceed to the next step;
(v) calculating the AR score of each input protein driving the cellular state “ASPC differentiation” and extracting those proteins which have maximum & positive (+1.44) and minimum & negative (−1.44) AR scores in the cellular state of “ASPC differentiation”;
(vi) repeating the simulation from step (iii) to (xix), if a protein shows wrong activity in any of the phenotype, otherwise proceed to the next step:
(a) declaring the proteins with positive (and maximum=+1.44) and negative (and minimum=−1.44) activity ratio (AR) score observed in a particular phenotype as the driver proteins for the development of that phenotype, for example, JAK2 and STAT3 proteins showing positive and maximum AR score are the driver of “ASPC differentiation” process and P53 protein having negative AR score=−1.44 is the driver of GSC renewal;
(b) renaming the simulation strategy from (xv)-(xix) as the model of “glioblastoma stem cells (GSC) development” model, in which the GSCs show continuous renewal process with no marker sign of apoptosis. GSCs also develop into matured but mutated (P53) astrocytes cells which clearly indicates the development of astrocytoma or glioma;
(c) fitting the distribution of the phenotype prediction scores of each cellular state observed in 100 simulation batches with different distribution functions and calculating the confidence interval of the phenotype predictor score of a cellular state from its corresponding distribution function;
(d) finding the cellular state Astrocytes progenitor cells (ASPC) differentiation and the AR scores of each protein which have shown either the value of +1.44 or −1.44; and
(e) removing the proteins with AR score either +1.44 or −1.44 from the input protein list made in step (v) and considering the P53 protein as constitutively down-regulated (binary: 0 or False) state in the logical equation model developed in step (iii) to create another new model.

7. The method of claim 1, wherein after determining the risk of tumor development by LGG or GBM from the general GBM developmental model by using the patient's mRNA sequencing data, the method of target screening and identifying the potential protein for personalized target-based therapy comprises:
(i) extracting the temporal activity profiles of intermediate molecules of the network model from the STG (a State Transition Graph) generated after simulation started from the initial states of the molecules to the cellular state representing high-grade (Grade-IV) glioblastoma cellular state;
(ii) extracting the activity profile of Grade-IV glioblastoma cellular state in STG;
(iii) identifying the delays and correlations of activity profiles of each intermediate molecule with the temporal activity profile observed for high-grade (Grade-IV) glioblastoma cellular state by Fast Fourier Transformation (FFT) analyses;
(iv) extracting the molecules having absolute correlation value ≥0.75 with P value <0.05, and Delay ≤3 with the activity pattern of high-grade (Grade-IV) glioblastoma cellular state, and selecting for perturbation analyses;
(v) perturbing the molecules by constitutively up-regulating molecules which have negative correlation with high-grade (Grade-IV) glioblastoma cellular state in the new perturbation analysis;
(vi) perturbing the molecules by constitutively down-regulating the molecules which have negative correlation with high-grade (Grade-IV) glioblastoma cellular state in the new perturbation analysis;
(vii) assessing the changes of frequencies of the cellular states representing the low-grade and high-grade glioblastoma cellular states in the perturbation studies with the previous simulation.
(viii) placing the perturbed molecules at the top of the list on the basis of its ability to maximum suppression of low-grade and high-grade glioblastoma cellular states in the perturbation study, wherein the molecules held rank at the top of the list are considered as potential targets for target-based, personalized glioblastoma therapy.
